(19) United States
(12) Patent Application Publication
(10) Pub. No.: US 2023/0048994 A1
(43) Pub. Date: Feb. 16, 2023

(54) RECOMBINANT AAV PRODUCTION

(71) Applicant: Asklepios Biopharmaceutical, Inc., Research Triangle Park, NC (US)

(72) Inventors: Jacob Smith, Research Triangle Park, NC (US); Josh Grieger, Research Triangle Park, NC (US)

(21) Appl. No.: 17/793,196

(22) PCT Filed: Jan. 15, 2021
(86) PCT No.: PCT/US2021/013689

§ 371 Date: Jul. 15, 2022

Related U.S. Application Data
(60) Provisional application No. 62/962,911, filed on Jan. 17, 2020.

(51) Int. Cl. C12N 15/86

(52) U.S. Cl. CPC C12N 15/86; C12N 2750/14143; C12N 2750/14151

(57) ABSTRACT

Methods for Producing Populations of High Titer Recombinant Adeno-Associated Virus (rAAV) Lacking Prokaryotic Sequences are disclosed.

### CROSS-REFERENCE TO RELATED APPLICATIONS

This application claims benefit under 35 U.S.C. § 119(e) of U.S. Provisional Application No. 62/962,911, filed Jan. 17, 2020, content of which is incorporated by reference in its entirety.

### FIELD OF THE DISCLOSURE

The present disclosure relates to the production of recombinant adeno-associated virus (rAAV) virions lacking prokaryotic sequences.

### BACKGROUND

Current methods to produce rAAV are still expensive despite years of research. Production rates of approximately 10⁵ genome copies (GC)/cell are now common, resulting in 10¹⁴ GC/L (Kotin RM. Large-scale recombinant adeno-associated virus production. Hum Mol Genet. 2011;20(R1):R2-R6. doi: 10.1093/hmg/ddr141). While this has proven to be sufficient to support early clinical trials, and could supply marketed product for small patient population indications, the deficiencies in scalability with this platform are a significant limitation (Clement N, Grieger JC. Manufacturing of recombinant adeno-associated viral vectors for clinical trials. Mol Ther Methods Clin Dev. 2016;3:16002. doi: 10.1038/mtm.2016.2; Wright JF. manufacturing and characterizing AAV-based vectors for use in clinical studies. Gene Ther. 2008;15(11):840-848. doi: 10.1038/gt.2008.65). As one could surmise, successfully delivering three plasmids to one cell is a relatively inefficient process. For larger-scale manufacturing efforts, transient delivery of plasmid requires excess quantities of DNA, adding to the overall cost of production and purification. Moreover, transient delivery of rep/cap genes in the presence of helper genes can also contribute to product heterogeneity, including AAV vectors lacking a transgene. These ‘empty capsids’ represent a significant proportion of virus produced in transient transfection assays. Thus, it is critically important to develop robust analytical quality control (QC) methods that will ensure similarities between production lots.

### SUMMARY

Embodiments of the invention are directed to closed linear large scale production of recombinant adeno-associated virus (rAAV) vectors lacking prokaryotic sequences.

In one aspect, provided herein is a method of producing a recombinant adeno-associated virus (rAAV). Generally, the method comprises transfecting a host cell line in a culture media with a) a nucleic acid sequence encoding helper proteins sufficient for rAAV replication; b) a nucleic acid sequence encoding AAV rep and AAV cap genes, and c) a close ended linear duplexed rAAV vector nucleic acid comprising at least one inverted terminal repeat (ITR) sequence and a heterologous transgene operably linked to one or more regulatory elements; incubating, for example, a transfected host cell line for a sufficient period of time to produce rAAV; optionally lysing the transfected host cells; and isolating/purifying the rAAV from the culture media. The method is amenable for producing high titers of rAAV. Thus, a titer of rAAV produced by the method can be higher than a titer of rAAV produced using a host cell line transfected with a corresponding amount of a plasmid DNA (pDNA) comprising the same heterologous transgene. In certain embodiments, total amount of nucleic acids from a), b), and c) per 1 x 10⁶ host cells is less than about 2 µg. In certain embodiments, the host cells are transfected using a transfection composition comprising a), b) and c), and a polycationic polymer, wherein a ratio of polycationic polymer to total amount of nucleic acid from a), b) and c), is from about 1:1 to about 3:1 (weight/weight). In some embodiments, the transfected host cell is lysed. In some other embodiments, the transfected cell is not lysed.

In certain embodiments, the titer of the rAAV produced by a method of the invention is higher than a titer of rAAV produced using a host cell line transfected with a corresponding amount of a plasmin DNA comprising the heterologous transgene. For example, the rAAV titer produced by a method of the invention is at least 1.25 fold, e.g., 1.5 fold, 1.75 fold, 2 fold, 2.25 fold, 2.5 fold, 2.75 fold, 3 fold, 3.5 fold, 4 fold, 4.5 fold, 5 fold or higher than the rAAV titer obtained with a corresponding amount of a plasmid DNA.

In certain embodiments, a method of producing a recombinant adeno-associated virus (rAAV) comprises providing a vector encoding for an AAV nucleic acid sequence or a closed linear AAV nucleic acid sequence; culturing a human embryonic cell line in suspension; transfecting the human cell line with the vector encoding the AAV nucleic acid sequence and a transfection composition or with the closed linear AAV nucleic acid sequence and a transfection composition; incubating the transfected human cell lines for between about 40 to 400 hours; optionally lysing the transfected human cell lines and purifying the nucleic acid sequences encoding the rAAV, thereby producing the rAAV. In some aspect of the embodiment, the transfected host cell is lysed. In some other embodiments, the transfected cell is not lysed.

In certain embodiments, the transfection composition comprises: (i) a vector encoding adenovirus helper proteins, (ii) a vector comprising an AAV rep gene and an AAV capsid (cap) protein gene, and (iii) a vector comprising AAV inverted terminal repeat (ITR) sequences. In some embodiments, at least one of vectors (i)-(iii) is comprised in a closed linear AAV nucleic acid sequence. For example, the vector (iii) is comprised in a close ended linear duplexed vector nucleic acid comprising at least one inverted terminal repeat (ITR) sequence and a heterologous transgene operably linked to one or more regulatory elements.

In certain embodiments, the vector encoding adenovirus helper proteins lacks Adenoviral structural and replication genes. In certain embodiments, the AAV rep and capsid (cap) genes are from different serotypes. In certain embodiments, the AAV rep and capsid genes from the same serotype. Examples of AAV serotypes include AAV1, AAV2, AAV3, AAV4, AAV5, AAV6, AAV7, AAV8, AAV9, AAV10, AAV11, AAV 13 and the like. In certain embodiments, the AAV rep gene is from a serotype selected from the group consisting of AAV2, 3, 8, 9 and 10. In certain embodiments, the AAV cap gene is from a serotype selected from the group consisting of AAV2, 3, 8, 9 and 10. For Example, the AAV rep gene is an AAV2 rep gene and the AAV capsid gene is an AAV8 capsid gene. In certain embodiments, the AAV inverted terminal repeat (ITR) sequences are adeno-associated virus 2 inverted terminal repeat (ITR) sequences. Virtually any other combination of serotypes can be used.

In certain embodiments, the rAAV particles have at least an AAV ITR from AAV serotypes selected from the group consisting of AAV1, 2, 3 (e.g., 3a, 3b), 4, 5, 6, 7, 8, 9, 10, 11, and 13. In certain embodiments, the AAV ITR is from a serotype selected from the group consisting of AAV2, 3 (e.g., 3a, 3b), 8, 9 and 10. In certain embodiments, the AAV ITR and the AAV cap genes are from different serotypes. In certain embodiments, the AAV ITR and AAV cap genes are from the same serotype. In some embodiments the AAV ITRs are wild type, mutant or synthetic. In certain embodiments the mutant ITRs comprise substation, addition and or deletion of one or more amino acids. In some embodiments, the AAV ITRs are the exemplary ITRs from US 7,790,154; US 8,361,457; US 8,784,799; US 9,447,433; US 9,169,494; or US 10,233,428.

In certain embodiments, the human embryonic cell line is suspension-adapted, serum-free cell line derived from a human embryonic kidney cell line.

In certain embodiments, the suspension of human embryonic cell line is progressively cultured in increasing volumes prior to transfection. In certain embodiments, the culturing volumes are progressively increased from about 50 ml volumes to about 100 liter volumes. In certain embodiments, the culture medium for progressive expansion of the human embryonic cell suspension from about the 50 ml up to about a 10 liter volume comprises a concentration of an amino acid from about 1 mM to about 20 mM. In certain embodiments, a culture medium having a volume of about 5 liters comprises a concentration of an amino acid of about 10 mM. In certain embodiments, the amino acid is L-glutamine. In certain embodiments, the culture medium having a volume of about 50 liters comprises: at least, from about 1 mM to about 20 mM L-glutamine, at least from about 0.01% to about 1% a nonionic, surfactant polyol or detergent and at least, from about 0.001% to about 1% of an anti-foaming agent. In certain embodiments, the nonionic, surfactant polyol comprises pluronic acid.

The cell density of the host cells can range from about 3.0 ×10⁶ to about 1 ×10⁸ viable cells/ml. For example, cell density of the host cells can range from about 3.5 × 10⁷ to about 8.5 × 10⁷ viable cells/ml. In certain embodiments, cell density of the host cells can range from 3 × 10⁶ to about 6 × 10⁶ viable cells/ml. For example, cell density of the host cells can be from about 4.0 ×10⁶ to about 6 ×10⁶ viable cells/ml. In certain embodiments, the cell density of the host cells can be about 2.5 × 10⁷ viable cells/ml. In some other embodiments, the cell density of the host cells in the cell culture can be about 3 × 10⁷ viable cells/ml.

In certain embodiments, the cultured human embryonic cell line comprises a cell density of about 3.0 ×10⁶ to about 1×10⁸ viable cells/ml. In one embodiment the cultured human embryonic cell line comprises 2.5 × 10⁷ viable cells/ml. In another embodiment, the cultured human embryonic cell line comprises 3 × 10⁷ viable cells/ml. In some embodiments, the cultured human embryonic cell line comprises a cell density of about 3.5 × 10⁷ to about 8.5 × 10⁷ viable cells/ml. In certain embodiments, the cultured human embryonic cell line comprises a cell density of about 4.0 ×10⁶ to about 6 ×10⁶ viable cells/ml. In certain embodiments, the human embryonic cell line is transfected with the vector encoding the AAV nucleic acid sequence and the transfection composition or with the closed linear AAV nucleic acid sequence and the transfection composition at a cell density from about 3 × 10⁶ to about 5 × 10⁶ viable cells/ml.

In certain embodiments, the transfection composition comprises at least about 5% volume/volume (v/v) to about 50% v/v of the culture medium. In certain embodiments, the transfection composition comprises at least about 5% volume/volume (v/v) to about 20% v/v of the culture medium. In certain embodiments, the transfection composition comprises at least about 10% volume/volume (v/v) to about 20% v/v of the culture medium In some embodiments, the transfection composition comprises at least about 5% volume/volume (v/v) to about 10% v/v of the culture medium. In certain embodiments, the transfection composition comprises about 1 liter to about 5 liters of medium In certain embodiments, the nucleic acid sequences added to the transfection composition comprise: about 0.1 µg to about 1 µg of Ad helper DNA, Rep/Cap DNA, or transgene per 0.5 ×10⁶ to about 5 ×10⁶ cells.

In certain embodiments, the method further comprises: (i) adding about 1 liter of medium to the transfected cells; and (ii) adding a cationic polymer at a ratio of between about 1:1 of polymer to DNA to about 3:1 of the polymer to DNA over a time course of about 1 to about 5 minute. In certain embodiments, the cationic polymer is added at a ratio of 2.2:1 of the polymer to DNA over a time course of about 1 minute. In certain embodiments, the cationic polymer comprises a fully hydrolyzed linear polyethylenimine (PEI).

In certain embodiments, the temperature of the culture medium comprising the host cells is increased to 37° C. at about 12 to 36 hours prior to transfection. For example, the temperature of the culture medium comprising the human embryonic cell suspension is increased to 37° C. at about 12 to 36 hours prior to transfection.

In certain embodiments, the culture medium is subjected to an air sparge at a flow rate between about 0.1 LPM to about 1.0 LPM. In certain embodiments, the culture medium is subjected to an air sparge at a flow rate of about 0.5 LPM. In certain embodiments, the culture medium is subjected to an air sparge at a flow rate of about 1.5 LPM, 2 LPM, 5 LPM, 7 LPM, 10 LPM, 15 LPM, 20 LPM, 30 LPM, 40 LPM, 50 LPM, 60 LPM, 70 LPM, 80 LPM< 90 LPM or 100 LPM. In certain embodiments, the culture medium is maintained at a pH of at least about 7.0.

In certain embodiments, a method of producing a population of high titer recombinant adeno-associated virus (rAAV) lacking prokaryotic sequences comprises transfecting a mammalian cell with a) a nucleic acid sequence encoding helper proteins sufficient for rAAV replication; b) a nucleic acid sequence encoding rep and cap genes, and c) a close ended linear duplexed rAAV vector nucleic acid comprising at least one ITR and a heterologous transgene operably linked to one or more regulatory elements, wherein the total amount of nucleic acid transfected from a), b), and c) per 1 × 10⁶ cells is less than 1 µg; culturing the transfected cells for at least 24 hours, e.g., at least 40 hours; optionally lysing the transfected cells and purifying the rAAV vector particles produced; wherein the titer of rAAV is at least 9.3 × 10¹³ vector genomes/3.0 × 10⁹ viable cells transfected. In some aspect of the embodiment, the transfected host cell is lysed. In some other embodiments, the transfected cell is not lysed.

In certain embodiments, the titer of the rAAV vector particles is at least 1 × 10¹⁰ to at least 1 × 10¹⁶ vector genomes/1.0 × 10⁸ to 1 × 10¹¹ viable cells transfected. In certain embodiments, the titer of rAAV vector particles is at least 1 × 10¹¹ vector genomes/1.0 × 10⁹ to 2 × 10¹¹ viable cells transfected. In certain embodiments, the titer of rAAV vector particles is at least 1 × 10¹² vector genomes/2.0 × 10⁹ to 3 × 10¹¹ viable cells transfected. In certain embodiments, the titer of rAAV vector particles is at least 1 × 10¹³ vector genomes/2.0 × 10⁹ to 2 × 10¹¹ viable cells transfected. In certain embodiments, the titer of rAAV vector particles is at least 1 × 10¹⁴ vector genomes/2.0 × 10⁹ to 4 × 10¹¹ viable cells transfected. In certain embodiments, the titer of rAAV vector particles is at least 2 × 10¹⁴ vector genomes/3.0 × 10⁹ to 5 × 10¹¹ viable cells transfected. In certain embodiments, the titer of rAAV vector particles is at least 3 × 10¹⁴ vector genomes/4.0 × 10⁹ to 5 × 10¹¹ viable cells transfected. In certain embodiments, the titer of rAAV vector particles is at least 4 × 10¹⁴

vector genomes/5.0 × 10⁹ to 5 × 10¹¹ viable cells transfected. In certain embodiments, the titer of rAAV vector particles is at least 5 × 10¹⁴ vector genomes/6.0 × 10⁹ to 5 × 10¹¹ viable cells transfected. In certain embodiments, the titer of rAAV vector particles is at least 1.25 × 10¹⁴ vector genomes/4.0 × 10⁹ viable cells transfected.

In certain embodiments, the titer of rAAV vector particles is at least 2 × 10¹¹ vp/ml. In certain embodiments, the titer of rAAV vector particles is at least 2.5 × 10¹¹ vp/ml. In certain embodiments, the titer of rAAV vector particles is at least 3 × 10¹¹ vp/ml. In certain embodiments, the titer of rAAV vector particles is at least 3.5 × 10¹¹ vp/ml. In certain embodiments, the titer of rAAV vector particles is at least 4 × 10¹¹ vp/ml. In certain embodiments, the titer of rAAV vector particles is at least 4.5 × 10¹¹ vp/ml. In certain embodiments, the titer of rAAV vector particles is at least 5 × 10¹¹ vp/ml. In certain embodiments, the titer of rAAV vector particles is at least 5.5 × 10¹¹ vp/ml. In certain embodiments, the titer of rAAV vector particles is at least 6 × 10¹¹ vp/ml. In certain embodiments, the titer of rAAV vector particles is at least 6.5 × 10¹¹ vp/ml. In certain embodiments, the titer of rAAV vector particles is at least 7 × 10¹¹ vp/ml. In certain embodiments, the titer of rAAV vector particles is at least 7.5 7× 10¹¹ vp/ml. In certain embodiments, the titer of rAAV vector particles is at least 8 × 10¹¹ vp/ml. In certain embodiments, the titer of rAAV vector particles is at least 8.5 × 10¹¹ vp/ml. In certain embodiments, the titer of rAAV vector particles is at least 9 × 10¹¹ vp/ml. In certain embodiments, the titer of rAAV vector particles is at least 9.5 × 10¹¹ vp/ml. In certain embodiments, the titer of rAAV vector particles is at least 1 × 10¹² vp/ml. In certain embodiments, the titer of rAAV vector particles is at least 5 × 10¹² vp/ml.

In certain embodiments of the present invention, the titer of rAAV vector particles obtained using closed linear (c1DNA) is at least 1e¹¹ vg/ml. In some embodiments, the titer of rAAV vector particles is at least 2e¹¹ vg/ml. In some embodiments, the titer of rAAV vector particles is at least 3e¹¹ vg/ml. In certain embodiments, the titer of rAAV vector particles is at least 4e¹¹ vg/ml. In certain embodiments, the titer of rAAV vector particles is at least 5e¹¹ vg/ml. In several embodiments, the titer of rAAV vector particle is at least 6e¹¹ vg/ml. In some embodiments, the titer of rAAV vector particle is at least 7e¹¹ vg/ml. In certain embodiments, the titer of the rAAV particle is at least 8e¹¹ vg/ml. In some embodiments, the titer of the rAAV particle is at least 8.5e¹¹ vg/ml. In other embodiments, the titer of the rAAV particle is at least 9e¹¹ vg/ml. In yet another embodiment, the titer of the rAAV particle is at least 9.5e¹¹ vg/ml. In certain embodiments, the titer of the rAAV particle is at least 1e¹² vg/ml.

In some embodiments, the rAAV vector particle obtained using closed linear (c1DNA) is about 2 to about 3 fold higher than that obtained using plasmid DNA (pDNA). In another embodiment, the rAAV vector particle obtained using closed linear (c1DNA) is about 4 to about 5 fold higher than that obtained using plasmid DNA (pDNA). In another embodiment, the rAAV vector particle obtained using closed linear (c1DNA) is about 6 to about 8 fold higher than that obtained using plasmid DNA (pDNA). In various embodiments, the rAAV vector particle obtained using closed linear (c1DNA) is about 9 to about 15 fold higher than that obtained using plasmid DNA (pDNA).

Certain embodiments of the methods described herein include use of a) a nucleic acid sequence encoding helper proteins, b) a nucleic acid sequence encoding rep and cap genes, and c) a close ended linear duplexed rAAV vector nucleic acid comprising at least one ITR and a heterologous transgene operably linked to one or more regulatory elements. The ratio of a) a nucleic acid sequence encoding helper proteins to b) a nucleic acid sequence encoding rep and cap genes to c) a close ended linear duplexed rAAV vector nucleic acid comprising at least one ITR and a heterologous transgene operably linked to one or more regulatory elements [a):b):c)] can be optimized for specific nucleic acids used. For example, the ratio of a):b):c) can be about 0.5-1.75: about 0.75-2.25: about 0.5-1.75 (weight: weight: weight). In certain embodiments, the ratio of a):b):c) is about 0.75-1.5: about 1-1.75: about 0.75-1.25 (weight: weight: weight).

In certain embodiments, the ratio of a) a nucleic acid sequence encoding helper proteins to b) a nucleic acid sequence encoding rep and cap genes to c) a close ended linear duplexed rAAV vector nucleic acid comprising at least one ITR and a heterologous transgene operably linked to one or more regulatory elements [a):b):c)] is about 1:about 1-1.6: about 1 (weight: weight: weight). In certain embodiments, the ratio of a):b):c) is about 0.5 : 1: 1 (weight: weight: weight). In certain embodiments, the ratio of a):b):c) is about 0.5 : 1: 0.5 (weight: weight: weight). In certain embodiments, the ratio of a):b):c) is about 0.75 : 1: 0.75 (weight: weight: weight). In certain embodiments, the ratio of a):b):c) is about 0.5 : 1: 1 (weight: weight: weight). In certain embodiments, the ratio of a):b):c) is about 0.5 : 1: 0.75 (weight: weight: weight).In certain embodiments, the ratio of a):b):c) is about 0.5 : 1.5 : 1 (weight: weight: weight). In certain embodiments, the ratio of a):b):c) is about 1 : 1.5 : 1 (weight: weight: weight). In certain embodiments, the ratio of a):b):c) is about 1 : 1.6 : 1 (weight: weight: weight). In certain embodiments, the ratio of a):b):c) is about 1 : 1.75 : 1 (weight: weight: weight). In certain embodiments, the ratio of a):b):c) is about 1 : 1.8 : 1 (weight: weight: weight). In certain embodiments, the ratio of a):b):c) is about 1 : 1.85 : 1 (weight: weight: weight). In certain embodiments, the ratio of a):b):c) is about 1 : 1.90 : 1 (weight: weight: weight). In certain embodiments, the ratio of a):b):c) is about 1 : 1.95 : 1 (weight: weight: weight). In certain embodiments, the ratio of a):b):c) is about 1 : 2 : 1 (weight: weight: weight). In certain embodiments, the ratio of a):b):c) is about 1.4: about 1.5: about 1 (weight: weight: weight).

In certain embodiments, the host cells are transfected using a transfection composition comprising a) a nucleic acid sequence encoding helper proteins, b) a nucleic acid sequence encoding rep and cap genes, c) a close ended linear duplexed rAAV vector nucleic acid comprising at least one ITR and a heterologous transgene operably linked to one or more regulatory elements, and d) a polycationic polymer. The cationic polymer can be any synthetic or natural polymer bearing at least two positive charges per molecule and having sufficient charge density and molecular size to bind to nucleic acid under physiological conditions. In certain embodiments, the polycationic polymer contains one or more amine residues, such as polyethylene imine (PFI) or a polyamino acid such as polyomithine, polyarginine, and polylysine. In preferred embodiments, the polycationic polymer is PEI.

The polycationic polymer can be any synthetic or natural polymer bearing at least two positive charges per molecule and having sufficient charge density and molecular size so as to bind to nucleic acid under transfectio conditions (i.e., pH and salt conditions encountered within a cell culture. Suitable cationic polymers include, for example, polyethylene imine (PEI), polyallylamine, polyvinylamine, polyvinylpyridine, aminoacetalized poly(vinyl alcohol), acrylic or methacrylic polymers (for example, poly(N,N-dimethylaminoethylmethacrylate)) bearing one or more amine residues, polyamino acids such as polyomithine, polyarginine, and polylysine, protamine, cationic polysaccharides such as chitosan, DEAE-cellulose, and DEAE-dextran, and polyamidoamine dendrimers (cationic dendrimer), as well as copolymers and blends thereof

Polycationic polymers can be either linear or branched, can be either homopolymers or copolymers, and when containing amino acids can have either L or D configuration, and can have any mixture of these features. Preferably, the cationic polymer molecule is sufficiently flexible to allow it to form a compact complex with one or more nucleic acid molecules.

The molecular weight of the polycationic polymer can be varied in view of the identity of the one or more nucleic acids. Accordingly, in some embodiments, the polycationic polymer has a molecular weight of between about 5,000 Daltons and about 100,000 Daltons, more preferably between about 5,000 and about 50,000 Daltons, most preferably between about 10,000 and about 35,000 Daltons

In certain embodiments, the polycationic polymer is polyethylenimine (PEI). For example, the polycationic polymer is a linear polyethylenimine. In certain embodiments, the polycationic polymer is fully hydrolyzed polyethylenimine.

In some embodiments, the polycationic polymer is a stable cationic polymer.

The ratio of the polycationic polymer to the total amount of nucleic acids from a), b) and c) can be varied for optimal transfection. For example, the ratio of the polycationic polymer to the total amount of nucleic acids from a), b) and c) can be from about 1.5:1 to about 2.75:1. In certain embodiments, the ratio of the polycationic polymer to the total amount of nucleic acids from a), b) and c) can be from about 1.9:1 to about 2.6:1. In certain embodiments, the ratio of the polycationic polymer to the total amount of nucleic acids from a), b) and c) can be from about 1:1.5 to about 1:2.75. For example, the ratio of the polycationic polymer to the total amount of nucleic acids from a), b) and c) can be from about 1:1.9 to about 1:2.6.

The polycationic polymer is present in the transfection composition in an amount effective to complex with the nucleic acids from a), b) and c) to form a complex. In certain embodiments, the relative amount of the polycationic polymer and the nucleic acids from a), b) and c) can be represented by the number of nitrogen atoms in the polycationic polymer divided by the number of phosphorous atoms in the nucleic acids (N/P ratio). In certain embodiments, the polycationic polymer and the nucleic acids from a), b) and c) are present at an N/P ratio of between about 2 and about 15, more preferably between about 3 and about 12, most preferably between about 4 and about 9.

In certain embodiments, the steps of a), b) and c) are transfected using a transfection composition comprising a), b) and c), and a stable cationic polymer, wherein the ratio of stable cationic polymer to total amount of nucleic acid from a), b) and c) is about 1.5:1. In certain embodiments, the ratio of stable cationic polymer to total amount of nucleic acid from a), b) and c), is about 0.5:1 (weight:weight). In certain embodiments, the ratio of stable cationic polymer to total amount of nucleic acid from a), b) and c), is about 0.75:1 (weight:weight). In certain embodiments, the ratio of stable cationic polymer to total amount of nucleic acid from a), b) and c), is about 1:1 (weight:weight). In certain embodiments, the ratio of stable cationic polymer to total amount of nucleic acid from a), b) and c), is about 1.75:1 (weight:weight). In certain embodiments, the ratio of stable cationic polymer to total amount of nucleic acid from a), b) and c), is about 2:1 (weight:weight), is about 2.2:1 (weight:weight). In certain embodiments, the ratio of stable cationic polymer to total amount of nucleic acid from a), b) and c), is about 2.5:1 (weight:weight). In certain embodiments, the ratio of stable cationic polymer to total amount of nucleic acid from a), b) and c), is about 3:1 (weight:weight). In certain embodiments, the ratio of stable cationic polymer to total amount of nucleic acid from a), b) and c), is about 1:0.75 (weight:weight). In certain embodiments, the ratio of stable cationic polymer to total amount of nucleic acid from a), b) and c), is about 1:0.5 (weight:weight). In certain embodiments, the ratio of stable cationic polymer to total amount of nucleic acid from a), b) and c), is about 1:0.25 (weight:weight).

In certain embodiments, each of a), b) and c) are provided on one or more close ended linear duplexed nucleic acid molecules. In certain embodiments, the transfected nucleic acids a), b), and c) are synthetic nucleic acids and devoid of prokaryotic cellular modifications of DNA. In certain embodiments, the transfected a), b) and c) are synthetic nucleic acids and devoid of eukaryotic and prokaryotic cellular modifications of DNA. In certain embodiments, the packaged nucleic acids in the purified recombinant AAV (rAAV) lacks prokaryotic and eukaryotic DNA sequences. In one embodiment, the non-AAV vector DNA makes up less than 10% of total DNA in the rAAV particle.

In certain embodiments, a method of producing a population of purified recombinant adeno-associated virus (rAAV) that lacks prokaryotic sequences, comprises: transfecting the mammalian cell line in suspended in culture medium with a transfection composition; wherein, the transfection composition comprises a) a nucleic acid sequence encoding helper proteins sufficient for rAAV replication ; b) a nucleic acid sequence encoding rep and cap genes, and c) a close ended linear duplexed rAAV vector nucleic acid comprising at least one ITR and a heterologous transgene operably linked to one or more regulatory elements, and d) a stable cationic polymer; and wherein, the ratio of the stable cationic polymer to the total amount of nucleic acid contents from a), b) and c) is at least 1.5:1; culturing the transfected cell line for at least 24 hours, e.g., at least 40 hours; lysing the transfected cell line of step ii); purifying the rAAV, wherein the purified virus has a particle to infectivity ratio of less than 2 × 10⁴ vg/TCID50. In some aspect of the embodiment, the transfected host cell is optionally lysed. In some embodiments, the purified recombinant AAV (rAAV) generated has a particle to infectivity ratio of less than 1.5×10⁴ vg/TCID50. In some embodiments, the purified recombinant AAV (rAAV) has a particle to infectivity ratio of less than 1×10⁴ vg/TCID50. In some embodiments, the purified recombinant AAV (rAAV) has a particle to infectivity ratio of less than 9×10³ vg/TCID50. In some embodiments, the purified recombinant AAV (rAAV) has a particle to infectivity ratio of less than 8×10³ vg/TCID50. In some embodiments, the purified recombinant AAV (rAAV) has a particle to infectivity ratio of less than 7×10³ vg/TCID50. In some embodiments, the purified recombinant AAV (rAAV) has a particle to infectivity ratio of less than 6×10³ vg/TCID50. In some embodiments, the purified recombinant AAV (rAAV) has a particle to infectivity ratio of less than 5×10³ vg/TCID50. In some embodiments, the purified recombinant AAV (rAAV) has a particle to infectivity ratio of less than 4×10³ vg/TCID50. In some embodiments, the purified recombinant AAV (rAAV) has a particle to infectivity ratio of less than 3×10³ vg/TCID50. In some embodiments, the purified recombinant AAV (rAAV) has a particle to infectivity ratio of less than 2×10³ vg/TCID50. In some embodiments, the purified recombinant AAV (rAAV) has a particle to infectivity ratio of less than 1×10³ vg/TCID50. In some embodiments, the purified recombinant AAV (rAAV) has a particle to infectivity ratio of less than 9×10² vg/TCID50. In some embodiments, the purified recombinant AAV (rAAV) has a particle to infectivity ratio of less than 8×10² vg/TCID50. In some embodiments, the purified recombinant AAV (rAAV) has a particle to infectivity ratio of less than 7×10² vg/TCID50. In some embodiments, the purified recombinant AAV (rAAV) has a particle to infectivity ratio of less than 6×10² vg/TCID50. In some embodiments, the purified recombinant AAV (rAAV) has a particle to infectivity ratio of less than 5×10² vg/TCID50.

In certain embodiments of any one of the aspects, the infectious particle titer is at least 1 × 10⁴ vg/TCID50. In certain embodiments, the infectious particle titer is at least 1.5 × 10⁴ vg/TCID50. In certain embodiments, the infectious particle titer is at least 2 × 10⁴ vg/TCID50. In certain embodiments, the infectious particle titer is at least 2.5 × 10⁴ vg/TCID50. In certain embodiments, the infectious particle titer is at least 3 × 10⁴ vg/TCID50. In certain embodiments, the infectious particle titer is at least 3.5 × 10⁴ vg/TCID50. In certain embodiments, the infectious particle titer is at least 4 × 10⁴ vg/TCID50. In certain embodiments, the infectious particle titer is at least 4.5 × 10⁴ vg/TCID50. In certain embodiments, the purified virus has a particle to infectivity ratio of at least 5 × 10⁴ vg/TCID50.

In some embodiments, the method of producing recombinant AAV involves transient transfection method. In some embodiments, the method of producing recombinant AAV involves stable transfection method. In various embodiments, the transfection is performed in suspension.

Embodiments of the methods described herein include incubating the inoculated cell culture medium, e.g., the transfected host cells for a period of time to produce rAAV. For example, the inoculated cell culture medium, e.g., the transfected host cells can be incubated for a period of at least 24 hours. For example, the transfected host cells can be incubated for a period of at least 30 hours. In certain embodiments, the inoculated cell culture medium, e.g., the transfected host cells are incubated between 30 to 100 hours, or, 30 to 150 hours, or, 30 to 200 hours, or, 40 to 100 hours, or, 40 to 150 hours, or, 40 to 200 hours, or, 40 to 300 hours, or, 40 to 350 hours, or, 40 to 400 hours, or, 40 to 450 hours or, 40 to 500 hours, or 40 to 550 hours, or 40 to 600 hours, or 40 to 650 hours, or, 40 to 700 hours, or, 40 to 750 hours, or, 40 to 800 hours, or, 40 to 850 hours, or, 40 to 900 hours, or, 40 to 950 hours, or, 40 to 1000 hours. In certain embodiments, the inoculated cell culture medium, e.g., the transfected cells are cultured between about 40 to 400 hours. In certain embodiments, the transfected cells are cultured between about 40 to 100 hours, or, about 40 to 150 hours, or, about 40 to 200 hours, or, about 40 to 250 hours, or, about 40 to 300 hours, or, about 40 to 350 hours, or, about 40 to 400 hours, or, about 40 to 450 hours, or, about 40 to 450 hours, or, about 40 to 500 hours, or, about 40 to 550 hours, or, about 40 to 600 hours, or, about 40 to 650 hours, or, about 40 to 700 hours, or, about 40 to 750 hours, or, about 40 to 800 hours, or, about 40 to 850 hours, or, about 40 to 900 hours, or, about 40 to 950 hours, or, about 40 to 1000 hours. In certain embodiments, the inoculated cell culture medium, e.g., the transfected cells are cultured for at least 24 hours or at least 30 hours or at least 40 hours or at least 45 hours or at least 50 hours or at least 55 hours or at least 60 hours or at least 65 hours or at least 70 hours or at least or at least 72 hours or at least 75 hours. In certain embodiments, the inoculated cell culture medium, e.g., the transfected cells are cultured for no more than 1000 hours, or no more than 950 hours, or no more than 900 hours, or no more than 850 hours, or no more than 800 hours, or no more than 750 hours, or no more than 700 hours, or no more than 650 hours, or no more than 600 hours, or no more than 550 hours, or no more than 500 hours, or no more than 450 hours, or no more than 400 hours, or no more than 350 hours, or no more than 300 hours, or no more than 250 hours, or no more than 200 hours, or no more than 150 hours, or no more than 100 hours.

In certain embodiments, the mammalian cell line is a suspension cell or cell line i.e. non-adherent cell or cell line and the cells are transfected in suspension. In certain embodiments, the cell line is derived from a human embryonic kidney 293 cell line (HEK 293). In certain embodiments, the human embryonic kidney cells lack an SV40 antigen or other transformation antigens. In certain embodiments, the mammalian cell line is a suspension adapted serum free cell line. In certain embodiments, the cell lines are derived from primary blood cells e.g. lymphocytes, monocytes, macrophages, granulocytes, dendritic cells, erythrocytes. In certain embodiments, the cell lines are derived from cell biopsies and include, for example, lymph node cells, bone marrow cells, cord blood cells. In certain embodiments, the cell lines are derived from circulating tumor cells. In certain embodiments, the cell lines are derived from blood cell lines, for example, Jurkat and Molt4 T cell lines, U937 and THP pro-monocytes cell lines, B cell hybridomas. In certain embodiments, the cell lines are derived from stem cells. In certain embodiments, the cell line used for production of recombinant AAV is a stable cell line.

In certain embodiments, the suspension of the mammalian cell line is progressively cultured in increasing volumes of culture medium prior to transfection.

The methods disclosed herein are scalable and can be applied to the efficient and scalable production of rAAV. In other words, the methods described herein can be used with volumes of few ml to volumes of thousands of liters. As such, the methods described can be used for the industrial scale production of therapeutic rAAV compositions. In certain embodiments, the volume of the cell culture comprising the host cells can be at least about 50 liters. For example, the cell culture volume can be from about 50 liters to about 4000 liters. In certain embodiments, the cell culture volume can be from about 50 liters to about 2000 liters. For example, the cell culture volume can be from about 50 liters to about 250 liters. In another non-limiting example, the cell culture volume can be from about 50 liters to about 100 liters.

The volume of the cell culture comprising the host cells can be increased prior to transfection. For example, the cell culture volume can be increased from about 10 to 20, 30, 40 or 50 ml volumes to about 4000 liter volumes. In certain embodiments, the cell culture volume can be increased from about 10 to 20, 30, 40 or 50 ml volumes to about 2000 liters. For example, the cell culture volume can be increased from about 10 to 20, 30, 40 or 50 ml volumes to about 250 liters. In another non-limiting example, the cell culture volume can be increased from about 10 to 20, 30, 40 or 50 ml volumes to about 50 liters or about 100 liters. In certain embodiments, the cell culture volume can be increased from about 100 liter volumes. In certain embodiments, the cell culture volume can be increased from about 50 ml volumes to about 50 liter volumes. In certain embodiments, the cell culture volume can be increased from about 50 ml volumes to about 10 liter volumes.

In certain embodiments, the culturing volumes are progressively increased from about 50 ml volumes to about 4000 liter volumes. In certain embodiments, the culturing volumes are progressively increased from about 50 ml volumes to about 2000 liter volumes. In certain embodiments, the culturing volumes are progressively increased from about 10 to 20, 30, 40 or 50 ml volumes to about 250 liter volumes. In certain embodiments, the culturing volumes are progressively increased from about 10 to 20, 30, 40 or 50 ml volumes to about 100 liter volumes. In certain embodiments, the culturing volumes are progressively increased from about 10 to 20, 30, 40 or 50 ml volumes to about 50 liter volumes. In certain embodiments, the culture medium for progressive expansion of the human embryonic cell suspension from about a 50 ml volume up to about a 50 liter volume comprises a concentration of an amino acid from about 1 mM to about 20 mM. In certain embodiments, the wherein the culture medium having a volume of about 5 liters comprises a concentration of an amino acid of about 10 mM. In certain embodiments, the amino acid is L-glutamine.

In certain embodiments, the packaged nucleic acid of the rAAV virion lacks prokaryotic DNA sequences.

Certain embodiments include a nucleic acid sequence encoding helper proteins sufficient for rAAV replication. Helper proteins sufficient for rAAV replication have been widely studied, and a number of adenovirus genes encoding helper protein functions are known. For example, proteins encoded by early adenoviral gene regions E1A (present, for example, in HEK 293 cells), E2A, E40rf6, VAI RNA, and optionally VAII RNA, and, optionally, E1B (also present in HEK 293 cells) are thought to participate in the rAAV replication process. Accordingly, in certain embodiments, the nucleic acid sequence encoding helper proteins sufficient for rAAV replication comprises a nucleotide sequence encoding an adenoviral (Ad) helper protein. For example, the nucleic acid sequence encoding helper proteins sufficient for rAAV replication comprises a nucleotide sequence encoding adenoviral helper proteins E2A and/or E4.

In certain embodiments, a) the nucleic acid sequence encoding helper proteins sufficient for rAAV replication is adenovirus (Ad) helper that comprises nucleic acids encoding adenoviral helper proteins E2A and E4.

In certain embodiments, the amount of total of DNA from a), b) and c) are about 1 to about 50 ug. In certain embodiments, the amount of total of DNA from a), b) and c) are about 1 to about 20 ug. In certain embodiments, the amount of total of DNA from a), b) and c) are about 1 to about 10 ug. In certain embodiments, the amount of total of DNA from a), b) and c) are about 1 to about 8 ug. In certain embodiments, the amount of total of DNA from a), b) and c) are about 1 to about 6 ug. In certain embodiments, the amount of total of DNA from a), b) and c) are about 1 to about 3 ug. In certain embodiments, the amount of total of DNA from a), b) and c) are optionally 0.4, 0.5, 0.6, 0.7, 0.8, 0.9, 1, 1.2, 1.4, 1.6 or 1.8 µg. In certain embodiments, the amount of total of DNA from a), b) and c) is 0.75 µg.

In certain embodiments, the total amount of nucleic acids from a) a nucleic acid sequence encoding helper proteins sufficient for rAAV replication; b) a nucleic acid sequence encoding AAV rep and AAV cap genes, and c) a close ended linear duplexed rAAV vector nucleic acid comprising at least one inverted terminal repeat (ITR) sequence and a heterologous transgene operably linked to one or more regulatory elements used for inoculating the cell culture, e.g., transfecting the host cell is less than about 2 µg per 1 ×10⁶ cells. For example, the total amount of nucleic acids from a), b) and c) is less than about 1.5 µg per 1 ×10⁶ cells. In certain embodiments, the total amount of nucleic acids from a), b) and c) is less than about 1 µg per 1 ×10⁶ cells, or less than about 0.75 µg per 1 ×10⁶ cells.

In certain embodiments, the total amount of nucleic acids from a), b) and c) is at least 0.25 µg per 1 ×10⁶ cells. For example, the total amount of nucleic acids from a), b) and c) is at least 0.5 µg per 1 ×10⁶ cells. In certain embodiments, the total amount of nucleic acids from a), b) and c) is from about 0.25 µg per 1 ×10⁶ cells to about 2 µg per 1 ×10⁶ cells. For example, the total amount of nucleic acids from a), b) and c) is from about 0.5 µg per 1 ×10⁶ cells to about 1.5 µg per 1 ×10⁶ cells. In certain embodiments, the total amount of nucleic acids from a), b) and c) is from about 0.5 µg per 1 ×10⁶ cells to about 0.75 µg per 1 ×10⁶ cells.

In certain embodiments, the infectious particle titer is at least 3 ×10⁹ TCID50/ml. In certain embodiments, the infectious particle titer is at least 1 × 10⁵ TCID50/ml (Median Tissue Culture Infectious Dose) to about 1 × 10¹¹ TCID50. In certain embodiments, the infectious particle titer is at least 2 × 10⁵ TCID50/ml. In certain embodiments, the infectious particle titer is at least 5 × 10⁵ TCID50/ml. In certain embodiments, the infectious particle titer is at least 7.5 × 10⁵ TCID50/ml. In certain embodiments, the infectious particle titer is at least 8 × 10⁵ TCID50/ml. In certain embodiments, the infectious particle titer is at least 8.5 × 10⁵ TCID50/ml. In certain embodiments, the infectious particle titer is at least 9 × 10⁵ TCID50/ml. In certain embodiments, the infectious particle titer is at least 9.5 × 10⁵ TCID50/ml. In certain embodiments, the infectious particle titer is at least 9.9 × 10⁵ TCID50/ml. In certain embodiments, the infectious particle titer is at least 1 × 10⁶ TCID50/ml. In certain embodiments, the infectious particle titer is at least 1 × 10⁶ TCID50/ml. In certain embodiments, the infectious particle titer is at least 2 x 10⁶ TCID50/ml. In certain embodiments, the infectious particle titer is at least 5 × 10⁶ TCID50/ml. In certain embodiments, the infectious particle titer is at least 7.5 × 10⁶ TCID50/ml. In certain embodiments, the infectious particle titer is at least 8 × 10⁶ TCID50/ml. In certain embodiments, the infectious particle titer is at least 8.5 × 10⁶ TCID50/ml. In certain embodiments, the infectious particle titer is at least 9 × 10⁶ TCID50/ml. In certain embodiments, the infectious particle titer is at least 9.5 × 10⁶ TCID50/ml. In certain embodiments, the infectious particle titer is at least 9.9 × 10⁶ TCID50/ml. In certain embodiments, the infectious particle titer is at least 1 × 10⁷ TCID50/ml. In certain embodiments, the infectious particle titer is at least 2 × 10⁷ TCID50/ml. In certain embodiments, the infectious particle titer is at least 5 × 10⁷ TCID50/ml. In certain embodiments, the infectious particle titer is at least 7.5 × 10⁷ TCID50/ml. In certain embodiments, the infectious particle titer is at least 8 × 10⁷ TCID50/ml. In certain embodiments, the infectious particle titer is at least 9 × 10⁷ TCID50/ml. In certain embodiments, the infectious particle titer is at least 9.9 × 10⁷ TCID50/ml. In certain embodiments, the infectious particle titer is at least 1 × 10^{g} TCID50/ml. In certain embodiments, the infectious particle titer is at least 2.5 × 10^{g} TCID50/ml. In certain embodiments, the infectious particle titer is at least 5 × 10⁸ TCID50/ml. In certain embodiments, the infectious particle titer is at least 7.5 × 10⁸ TCID50/ml. In certain embodiments, the infectious particle titer is at least 8 × 10⁸ TCID50/ml. In certain embodiments, the infectious particle titer is at least 8.5 × 10⁸ TCID50/ml. In certain embodiments, the infectious particle titer is at least 9 × 10⁸ TCID50/ml. In certain embodiments, the infectious particle titer is at least 9.5 × 10⁸ TCID50/ml. In certain embodiments, the infectious particle titer is at least 9.9 × 10⁸ TCID50/ml. In certain embodiments, the infectious particle titer is at least 0.5 × 10⁹ TCID50/ml. In certain embodiments, the infectious particle titer is at least 1 × 10⁹ TCID50/ml. In certain embodiments, the infectious particle titer is at least 1.5 × 10⁹ TCID50/ml. In certain embodiments, the infectious particle titer is at least 2 × 10⁹ TCID50/ml. In certain embodiments, the infectious particle titer is at least 2.5 × 10⁹ TCID50/ml. In certain embodiments, the infectious particle titer is at least 3 × 10⁹ TCID50/ml. In certain embodiments, the infectious particle titer is at least 3.5 × 10⁹ TCID50/ml. In certain embodiments, the infectious particle titer is at least 4 × 10⁹ TCID50/ml. In certain embodiments, the infectious particle titer is at least 4.5 × 10⁹ TCID50/ml. In certain embodiments, the infectious particle titer is at least 5 × 10⁹ TCID50/ml. In certain embodiments, the infectious particle titer is at least 5.5 × 10⁹ TCID50/ml. In certain embodiments, the infectious particle titer is at least 6 × 10⁹ TCID50/ml. In certain embodiments, the infectious particle titer is at least 6.5 × 10⁹ TCID50/ml. In certain embodiments, the infectious particle titer is at least 7 × 10⁹ TCID50/ml. In certain embodiments, the infectious particle titer is at least 7.5 × 10⁹ TCID50/ml. In certain embodiments, the infectious particle titer is at least 8 × 10⁹ TCID50/ml. In certain embodiments, the infectious particle titer is at least 8.5 × 10⁹ TCID50/ml. In certain embodiments, the infectious particle titer is at least 9 × 10⁹ TCID50/ml. In certain embodiments, the infectious particle titer is at least 9.5 × 10⁹ TCID50/ml. In certain embodiments, the infectious particle titer is at least 9.9 × 10⁹ TCID50/ml. In certain embodiments, the infectious particle titer is at least 1 × 10¹⁰ TCID50/ml. In certain embodiments, the infectious particle titer is at least 2 × 10¹⁰ TCID50/ml. In certain embodiments, the infectious particle titer is at least 5 × 10¹⁰ TCID50/ml. In certain embodiments, the infectious particle titer is at least 7.5 × 10¹⁰ TCID50/ml. In certain embodiments, the infectious particle titer is at least 8 × 10¹⁰ TCID50/ml. In certain embodiments, the infectious particle titer is at least 8.5 × 10¹⁰ TCID50/ml. In certain embodiments, the infectious particle titer is at least 9 × 10¹⁰ TCID50/ml. In certain embodiments, the infectious particle titer is at least 9.5 × 10¹⁰ TCID50/ml. In some embodiments, the infectious titer TCID50/ml is preferably normalized to vg/ml. In some embodiments, the infectious particle titer is at least 10¹¹ TCID50/ml.

In certain embodiments, a method of large-scale production of a recombinant adeno-associated virus (rAAV) comprises providing a vector encoding for an AAV nucleic acid sequence or a closed linear AAV nucleic acid sequence; culturing a human embryonic cell line in suspension; transfecting the human cell line with the vector encoding the AAV nucleic acid sequence and a transfection composition or with the closed linear AAV nucleic acid sequence and a transfection composition; incubating the transfected human cell lines for between about 30 to 250 hours; lysing the transfected human cell lines and purifying the nucleic acid sequences, encoding the rAAV, thereby providing a large-scale production of rAAV. In certain embodiments, the transfected cells are incubated between 30 to 100 hours, or, 30 to 150 hours, or, 30 to 200 hours, or, 40 to 100 hours, or, 40 to 150 hours, or, 40 to 200 hours, or, 40 to 300 hours, or, 40 to 350 hours, or, 40 to 400 hours, or, 40 to 450 hours or, 40 to 500 hours, or 40 to 550 hours, or 40 to 600 hours, or 40 to 650 hours, or, 40 to 700 hours, or, 40 to 750 hours, or, 40 to 800 hours, or, 40 to 850 hours, or, 40 to 900 hours, or, 40 to 950 hours, or, 40 to 1000 hours.

In certain embodiments, the AAV Rep genes and the AAV Cap genes are from the same AAV serotype. In certain embodiments, the AAV Rep genes and the AAV Cap genes are from different AAV serotypes.

In certain embodiments, the human embryonic cell line is suspension-adapted, serum-free cell line derived from a human embryonic kidney cell line. The suspension of human embryonic cell line is progressively cultured in increasing volumes prior to transfection. In certain aspects, the culturing volumes are progressively increased from about 50 ml volumes to about 100 liter volumes. In certain embodiments, the culture medium for progressive expansion of the human embryonic cell suspension comprises a concentration of L-glutamine from about 1 mM to about 20 mM in culture medium volumes of about 50 ml up to about a 10 liter volume. In certain embodiments, the culture medium having a volume of about 5 liters comprises a concentration of L-glutamine of about 10 mM. In certain embodiments, the culture medium having a volume of about 50 liters comprises: at least, from about 1 mM to about 20 mM L-glutamine, at least from about 0.01% to about 1% pluronic acid and at least, from about 0.001% to about 1% of an anti-foaming agent.

In certain embodiments, the cultured human embryonic cell line comprises a cell density of about 3.0 ×10⁶ to about 1 × 10⁸ viable cells/ml. In certain aspects, the cultured human embryonic cell line comprises a cell density of about 4.0 ×10⁶ to about 6 × 10⁶ viable cells/ml. In certain embodiments, the human embryonic cell line is transfected with the vector encoding the AAV nucleic acid sequence and the transfection composition or with the closed linear AAV nucleic acid sequence and the transfection composition at a cell density from about 3 × 10⁶ to about 5 × 10⁶ viable cells/ml³.

In certain embodiments, the transfection composition comprises: (i) a vector encoding adenovirus helper proteins, (ii) a vector comprising an AAV rep gene and an AAV capsid (cap) protein gene, and (iii) a vector comprising AAV inverted terminal repeat (ITR) sequences. In certain embodiments, the vector encoding adenovirus helper proteins lacks Adenoviral structural and replication genes. In certain aspects, the AAV rep and capsid genes are different serotypes or are of the same serotype. In certain aspects, the AAV rep gene is an AAV2 rep gene and the AAV capsid gene is an AAV8 capsid gene. In certain aspects, the AAV inverted terminal repeat (ITR) sequences are adeno-associated virus 2 inverted terminal repeat (ITR) sequences. In certain embodiments, the nucleic acid sequences added to the transfection composition comprise: about 0.1 µg to about 1 µg of Ad helper DNA, Rep/Cap DNA, or transgene per 0.5 ×10⁶ to about 5 ×10⁶ cells. In some embodiments, the transfection is performed over a time course of about 10 minutes to about 60 minutes. In certain embodiments, the transfection is performed over a time course of about 10 minutes to about 120 minutes.

In certain embodiments, the cell density at transfection is about 2.0 × 10⁴ to about 1.0 × 10⁸ viable cells/ml. In certain embodiments, the cell density at transfection is about 5.0 × 10⁴ to about 5.0 × 10⁷ viable cells/ml. In certain embodiments, the cell density at transfection is about 1.0 × 10⁵ to about 1 × 10⁷ viable cells/ml. In certain embodiments, the cell density at transfection is about 5.0 × 10⁵ to about 1 × 10⁷ viable cells/ml. In certain embodiments, the cell density at transfection is about 2.0 × 10⁵ to about 9 × 10⁶ viable cells/ml. In certain embodiments, the cell density at transfection is about 1.0 × 10⁶ to about 7.5 × 10⁶ viable cells/ml. In certain embodiments, the cell density at transfection is about 1.0 × 10⁶ to about 7 × 10⁶ viable cells/ml. In certain embodiments, the cell density at transfection is about 1.0 × 10⁶ to about 5 × 10⁶ viable cells/ml. In certain embodiments, the cell density at transfection is about 1.0 × 106 to about 4 × 10⁶ viable cells/ml.

Certain embodiments include a transfection composition for transfecting the host cells. Generally, the transfection composition comprises a) a nucleic acid sequence encoding helper proteins sufficient for rAAV replication; b) a nucleic acid sequence encoding AAV rep and AAV cap genes, c) a close ended linear duplexed rAAV vector nucleic acid comprising at least one inverted terminal repeat (ITR) sequence and a heterologous transgene operably linked to one or more regulatory elements, and d) a polycationic polymer. In addition to the nucleic acids a), b) and c), the transfection composition can also comprise cell culture medium, i.e., medium used for the host cells. The transfection composition can have a volume of from about 5% to about 20% (volume/volume) of the host cell line culture volume. For example, the host cell line is transfected with a transfection composition volume of about 7.5% to about 15% (volume/volume) of the host cell line culture volume.

In certain embodiments, the transfection composition comprises at least about 5% volume/volume (v/v) to about 20% v/v of the culture medium. In certain embodiments, the transfection composition comprises about 1 liter to about 5 liters of medium Once the cells are transfected, about 1 liter of medium is added the transfected cells. In certain embodiments, a fully hydrolyzed linear polyethylenimine (PEI) is added at a ratio of between about 1:1 (PEI:DNA) to about 3:1 (PEI:DNA) over a time course of about 1 to about 5 minutes. In certain embodiments, fully hydrolyzed linear polyethylenimine (PEI) is added at a ratio of 2.2:1 (PEI:DNA) over a time course of about 1 minute. In certain aspects the transfected cell suspension is incubated for about 1-20 minutes before being transferred to a large volume bioreactor. In certain embodiments, the transfection-cell suspension is incubated for about three hours and quenched by a 10% (v/v) volume of chemically defined, serum-free media supplemented with about 10 mM L-Glutamine. In certain embodiments, the temperature of the culture medium comprising the human embryonic cell suspension is increased to 37° C. at about 12 to 36 hours prior to transfection. In certain embodiments, the culture medium is subjected to an air sparge at a flow rate between about 0.1 LPM to about 1.0 LPM. In certain embodiments, the culture medium is subjected to an air sparge at a flow rate of about 0.5 LPM. In certain embodiments, the culture medium is maintained at a pH of at least about 7.0.

In certain embodiments, a recombinant adeno-associated virus (rAAV) comprises a protelomerase target sequence. In certain aspects, the protelomerase target sequence comprises a double stranded palindromic sequence of at least 10 base pairs in length. In certain embodiments, the rAAV comprises a transgene.

In certain embodiments, a pharmaceutical composition comprises a closed linear recombinant adeno-associated virus (rAAV). In certain embodiments, the rAAV comprises a transgene.

In certain embodiments, the rAAV particles have an AAV capsid gene from an AAV serotype comprising AAV-1, AAV-2, AAV-3, AAV-4, AAV-5, AAV-6, AAV-7, AAV-8, AAV-9, AAV-10, AAV-11, AAV-12, AAV-13, AAV-14, AAV-15 and AAV-16. In certain embodiments, the rAAV particles comprise an AAV rep gene from an AAV serotype comprising AAV-1, AAV-2, AAV-3, AAV-4, AAV-5, AAV-6, AAV-7, AAV-8, AAV-9, AAV-10, AAV-11, AAV-12, AAV-13, AAV-14, AAV-15 and, AAV-16. In some embodiments, without limiting to, the rAAV particles are the exemplary rAAVs from U.S. Pat. 10,550,405, published international application WO2018170310A1, U.S. Pat. 7,892,809, U.S. Pat. 6,491,907, or U.S. Pat. 7,172,893. In certain embodiments, rAAV is a hybrid AAV comprising ITR from a certain AAV serotype and the capsid from a different AAV serotype. In some embodiments rAAVs can comprise rAAV virion. In certain embodiments rAAV capsids comprise substitution, addition and/or deletion of one or more amino acids; for example, capsids comprising inserted peptides for targeting.

### BRIEF DESCRIPTION OF THE DRAWINGS

FIG. 1 is a graph showing a comparison of vector genome titer in cell lysate expressed in vg/mL yields for a plasmid-based (AskBio) system (N=2) and favorable transfection conditions using clDNA system (N=5). The plots represent the mean +/- one standard deviation.

FIG. 2 is a plot of a prediction profiler which demonstrates that both µg clDNA/1E6 cells and PEI:DNA ratio have a statistically significant impact on Vg titer. p ≤0.0066.

FIG. 3 is a contour plot displaying the effects of clDNA and PEI:DNA ratio on vector genome titer in cell lysate, expressed in vg/mL.

FIG. 4 is a plot showing the effects of clDNA and PEI:DNA ratio on virus titer, expressed in vp/mL.

FIG. 5 is a plot of a prediction profiler showing that PEI:DNA ratio but not the amount of clDNA have a statistically significant impact on AAHrh10 CYP titer yield expressed in vp/mL.

FIG. 6 is a plot of a prediction profiler showing that both µg clDNA/1E6 cells and PEI:DNA ratio have a statistically significant impact on AAV8 GAA titer yield expressed in vp/ml.

### DETAILED DESCRIPTION

AAV is a protein shell surrounding and protecting a small, single-stranded DNA genome of approximately 4.8 kilobases (kb). AAV belongs to the parvovirus family and is dependent on co-infection with other viruses, mainly adenoviruses, in order to replicate. Initially distinguished serologically, molecular cloning of AAV genes has identified hundreds of unique AAV strains in numerous species. Its single-stranded genome contains three genes, Rep (Replication), Cap (Capsid), and aap (Assembly). These three genes give rise to at least nine gene products through the use of three promoters, alternative translation start sites, and differential splicing. These coding sequences are flanked by inverted terminal repeats (ITRs) that are required for genome replication and packaging. The Rep gene encodes four proteins (Rep78, Rep68, Rep52, and Rep40), which are required for viral genome replication and packaging, while Cap expression gives rise to the viral capsid proteins (VP; VP1/VP2/VP3), which form the outer capsid shell that protects the viral genome, as well as being actively involved in cell binding and internalization (Samulski RJ, Muzyczka N. AAV-mediated gene therapy for research and therapeutic purposes. Annu Rev Virol. 2014;1(1):427-451. doi: 10.1146/annurev-virology-031413-085355). It is estimated that the viral coat is comprised of 60 proteins arranged into an icosahedral structure with the capsid proteins in a molar ratio of 1:1:10 (VP1:VP2:VP3). The aap gene encodes the assembly-activating protein (AAP) in an alternate reading frame overlapping the cap gene. This nuclear protein is thought to provide a scaffolding function for capsid assembly (Naumer M, et al., J Virol. 2012;86(23):13038-13048. doi: 10.1128/JVI.01675-12). While AAP is essential for nucleolar localization of VP proteins and capsid assembly in AAV2, the subnuclear localization of AAP varies among 11 other serotypes and is nonessential in AAV4, AAV5, and AAV11 (Earley LF, et al. Adeno-associated Virus (AAV) assembly-activating protein is not an essential requirement for capsid assembly of AAV serotypes 4, 5, and 11. J Virol. 2017;91(3):1-21. doi:10.1128/jvi.01980-16).

In the examples section, which follows, compositions and methods for the large scale production of rAAV are described in detail. In certain embodiments, a method of large-scale production of a recombinant adeno-associated virus (rAAV) comprises providing a vector encoding for an AAV nucleic acid sequence or a closed linear AAV nucleic acid sequence; culturing a human embryonic cell line in suspension; transfecting the human cell line with the vector encoding the AAV nucleic acid sequence and a transfection composition or with the closed linear AAV nucleic acid sequence and a transfection composition; incubating the transfected human cell lines for between about 50 to 100 hours; harvesting the transfected human cell lines and purifying the rAAV vector, thereby providing a large-scale production of rAAV. In certain embodiments, the rAAV produced is a closed linear rAAV.

Accordingly, in certain embodiments, a method of producing a population of high titer recombinant adeno-associated virus (rAAV) lacking prokaryotic sequences comprises transfecting a mammalian cell with a) a nucleic acid sequence encoding helper proteins sufficient for rAAV replication; b) a nucleic acid sequence encoding rep and cap genes, and c) a close ended linear duplexed rAAV vector nucleic acid comprising at least one ITR and a heterologous transgene operably linked to one or more regulatory elements, wherein the total amount of nucleic acid transfected from a), b), and c) per 1 × 10⁶ cells is less than 1 µg; culturing the transfected cells for at least 40 hours; harvesting the transfected cells and purifying the rAAV vector particles produced; wherein the titer of rAAV is at least 9.3 × 10¹³ vector genomes/3.0 × 10⁹ viable cells transfected.

AAV sequences may be obtained from a variety of sources. For example, a suitable AAV sequence may be obtained as described in WO 2005/033321 or from known sources, e.g., the American Type Culture Collection, or a variety of academic vector core facilities. Alternatively, suitable sequences are synthetically generated using known techniques with reference to published sequences.

The AAV cap and rep sequences may be independently selected from different AAV parental sequences and be introduced into the host cell in a suitable manner known to one in the art. In certain embodiments, the rAAV particles have an AAV capsid gene from an AAV serotype comprising AAV-1, AAV-2, AAV-3, AAV-4, AAV-5, AAV-6, AAV-7, AAV-8, AAV-9, AAV-10, AAV-11, AAV-12, AAV-13. In certain embodiments, the rAAV particles have an AAV capsid gene from an AAV serotype selected from the group consisting of AAV2, 3, 8, 9 and 10.

In certain embodiments, the rAAV particles comprise an AAV rep gene from an AAV serotype comprising AAV-1, AAV-2, AAV-3, AAV-4, AAV-5, AAV-6, AAV-7, AAV-8, AAV-9, AAV-10, AAV-11, AAV-12, AAV-13. In certain embodiments, the rAAV particles have an AAV rep gene from an AAV serotype selected from the group consisting of AAV2, 3, 8, 9 and 10

The disclosure also provides for a method of producing a population of purified recombinant adeno-associated virus (rAAV) that lacks prokaryotic sequences, comprises: transfecting the mammalian cell line in suspended in culture medium with a transfection composition; wherein, the transfection composition comprises a) a nucleic acid sequence encoding helper proteins sufficient for rAAV replication ; b) a nucleic acid sequence encoding rep and cap genes, and c) a close ended linear duplexed rAAV vector nucleic acid comprising at least one ITR and a heterologous transgene operably linked to one or more regulatory elements, and d) a stable cationic polymer; and wherein, the ratio of the stable cationic polymer to the total amount of nucleic acid contents from a), b) and c) is at least 1.5:1; culturing the transfected cell line for at least 40 hours; harvesting the transfected cell line of step ii); purifying the rAAV, wherein the purified virus has a particle to infectivity ratio is less than 2 x 10⁴ vg/TCID50 and lacks prokaryotic DNA.

The mammalian cell lines used in embodiments of the invention include a suspension cell or cell line i.e. non-adherent cell or cell line. In certain embodiments, the cell line is derived from a human embryonic kidney cell line. In certain embodiments, the human embryonic kidney cells lack an SV40 antigen or other transformation antigens. In certain embodiments, the mammalian cell line is a suspension adapted serum free cell line. In certain embodiments, the cell lines are derived from primary blood cells, e.g. lymphocytes, monocytes, macrophages, granulocytes, dendritic cells, erythrocytes. In certain embodiments, the cell lines are derived from cell biopsies and include, for example, lymph node cells, bone marrow cells, cord blood cells. In certain embodiments, the cell lines are derived from circulating tumor cells. In certain embodiments, the cell lines are derived from blood cell lines, for example, Jurkat and Molt4 T cell lines, U937 and THP pro -monocytes cell lines, B cell hybridomas. In certain embodiments, the cell lines are derived from stem cells.

A viral cell culture utilizes cells containing, either stably or transiently, at least the minimum components required to generate an AAV particle. The minimum required components include, an expression cassette to be packaged into the AAV capsid, an AAV cap, and an AAV rep or a functional fragment thereof, and helper functions.

The cell also requires helper functions in order to package the AAV of the invention. Optionally, these helper functions may be supplied by a herpesvirus. In another embodiment, the necessary helper functions are each provided from a human or non-human primate adenovirus source, such as are available from a variety of sources, including the American Type Culture Collection (ATCC), Manassas, Va. (US). The sequences of a variety of suitable adenoviruses have been described. See, e.g., chimpanzee adenovirus C1 and C68 [U.S. Pat. No. 6,083,716]; Pan 5, Pan6 and Pan7, [WO 02/33645], hybrid adenoviruses such as those described [e.g., WO 05/001103], and GenBank.

A variety of suitable cells and cell lines have been described for use in production of AAV. The cell itself may be selected from any biological organism, including prokaryotic (e.g., bacterial) cells, and eukaryotic cells, including, insect cells, yeast cells and mammalian cells. Particularly desirable host cells are selected from among any mammalian species, including, without limitation, cells such as A549, WEHI, 3T3, 10T1/2, BHK, MDCK, , COS 1, COS 7, BSC 1, BSC 40, BMT 10, VERO, WI38, HeLa, a HEK 293 cell (which express functional adenoviral El), Saos, C2C12, L cells, HT1080, HepG2, Sf"c9, Sf-21, Tn368, BTI-Tn-5B1-4 (High-Five) and primary fibroblast, hepatocyte and myoblast cells derived from mammals including human, monkey, mouse, rat, rabbit, and hamster. The selection of the mammalian species providing the cells is not a limitation of this invention; nor is the type of mammalian cell, i.e., fibroblast, hepatocyte, tumor cell, etc.

In certain embodiments of any one of the aspects described herein, the host cell line is derived from a human embryonic kidney 293 cell line (HEK 293).

The host cell can contain at least the minimum adenovirus DNA sequences necessary to express an E1A gene product, an E1B gene product, an E2A gene product, and/or an E4 ORF6 gene product. The host cell may contain other adenoviral genes such as VAI RNA, but these genes are not required. The cell does not carry any adenovirus gene other than E1, E2A and/or E4 ORF6; does not contain any other virus gene which could result in homologous recombination of a contaminating virus during the production of rAAV; and it is capable of infection or transfection.

In certain embodiments of any one of the aspects, the host cells lack an SV40 antigen or other transformation antigens. For example, when human embryonic kidney cells, e.g., HEK 293 cells are used as host cell, such cells may lack an SV40 antigen or other transformation antigens.

Another type of host cell is one that is stably transformed with the sequences encoding rep and cap, and which is transfected with the adenovirus E1, E2A, and E4ORF6 DNA and a construct carrying the expression cassette as described above. Stable rep and/or cap expressing cell lines, such as B-50 (International Pat. Application Publication No. WO 99/15685), or those described in U.S. Pat. No. 5,658,785, may also be similarly employed. Another desirable host cell contains the minimum adenoviral DNA which is sufficient to express E4 ORF6. Yet other cell lines can be constructed using the novel modified cap sequences of the invention.

The preparation of a host cell involves techniques such as assembly of selected DNA sequences. This assembly may be accomplished utilizing conventional techniques. Such techniques include cDNA and genomic cloning, which are well known and are described in Sambrook et al., Molecular Cloning: A Laboratory Manual, Cold Spring Harbor Press, Cold Spring Harbor, N.Y., including polymerase chain reaction, synthetic methods, and any other suitable methods which provide the desired nucleotide sequence.

In certain embodiments, the host cell is a mammalian cell, i.e., the host cell line is a mammalian cell line. For example, the host cell, i.e., the host cell line, is human cell, such as a human embryonic cell line. In certain embodiments of any one of the aspects described herein, the host cell line is a human embryonic kidney cell line.

Cell culture work involved in rAAV production including expansion, seeding and transfection of adherent cells is cumbersome and resource intensive. Therefore, using cells suspended in aqueous liquid medium (“suspension cells”) for rAAV vector production is desirable due to its scalability and cost effectiveness. Accordingly, in certain embodiments of any one of the aspects described herein, the host cell line can be suspension-adapted. For example, host cells can be transfected with the nucleic acid vector(s) in suspension.

Components for AAV production (e.g., adenovirus E1a, E1b, E2a, and/or E4ORF6 gene products, rep or a fragment(s) thereof, cap, the expression cassette, as well as any other desired helper functions), may be delivered to the packaging host cell separately, or in combination, in the form of any genetic element which transfer the sequences carried thereon. As used herein, a genetic element (vector) includes, e.g., naked DNA, a plasmid, phage, transposon, cosmid, episome, a protein in a non-viral delivery vehicle (e.g., a lipid -based carrier), virus, etc., which transfers the sequences carried thereon. The selected vector may be delivered by any suitable method, including transfection, electroporation, liposome delivery, membrane fusion techniques, high velocity DNA-coated pellets, viral infection and protoplast fusion. The methods used to construct any embodiment of this invention are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y. See, e.g., K. Fisher et al, J Virol., 70:520-532 (1993) and U.S. Pat. No. 5,478,745.

The one or more of the adenoviral genes can be stably integrated into the genome of the host cell or stably expressed as episomes. The promoters for each of the adenoviral genes may be selected independently from a constitutive promoter, an inducible promoter or a native adenoviral promoter. The promoters, for example, may be regulated by a specific physiological state of the organism or cell (i.e., by the differentiation state or in replicating or quiescent cells) or by exogenously added factors. Examples of such factors include without limitation, antibiotics, cytokines, growth factors, hormones and the like.

In one embodiment, a stable or transient host cell will contain the required component(s) under the control of an inducible or regulatable promoter. However, the required component(s) may be under the control of a constitutive promoter or a synthetic promoter.

Regulatable promoters allow control of gene expression by exogenously supplied compounds, environmental factors such as temperature, or the presence of a specific physiological state, e.g., acute phase, a particular differentiation state of the cell, or in replicating cells only. Regulatable promoters and systems are available from a variety of commercial sources, including, without limitation, Invitrogen, Clontech and Ariad. Many other systems have been described and can be readily selected by one of skill in the art. Examples of promoters regulated by exogenously supplied promoters include the zinc -inducible sheep metallothionine (MT) promoter, the dexamethasone (Dex)-inducible mouse mammary tumor virus (MMTV) promoter, the T7 polymerase promoter system [WO 98/10088]; the ecdysone insect promoter [No et al., Proc. Natl. Acad. Sci. USA, 93:3346-3351 (1996)], the tetracycline-repressible system [Gossen et al., Proc. Natl. Acad. Sci. USA, 89:5547-5551 (1992)], the tetracycline-inducible system [Gossen et al., Science, 268:1766-1769 (1995), see also Harvey et al., Curr. Opin. Chem. Biol., 2:512-518 (1998)], the RU486-inducible system [Wang et al., Nat. Biotech., 15:239-243 (1997) and Wang et al., Gene Ther., 4:432-441 (1997)] and the rapamycin-inducible system [Magari et al., J Clin. Invest., 100:2865-2872 (1997)]. Still other types of inducible promoters which may be useful in this context are those which are regulated by a specific physiological state, e.g., temperature, acute phase, a particular differentiation state of the cell, or in replicating cells only.

In certain instances, a native promoter is used. The native promoter may be used when it is desired that expression of the gene product should mimic the native expression. The native promoter may be used when expression of a desired transgene must be regulated temporally or developmentally, or in a tissue-specific manner, or in response to specific transcriptional stimuli. Other native expression control elements, such as enhancer elements, polyadenylation sites or Kozak consensus sequences may also be used to mimic the native expression.

In cases where a transgene is included, the transgene operably linked to a tissue-specific promoter. For instance, if expression in skeletal muscle is desired, a promoter active in muscle should be used. These include non-limiting examples of promoters from genes encoding skeletal β-actin, myosin light chain 2A, dystrophin, muscle creatine kinase, as well as synthetic muscle promoters with activities higher than naturally-occurring promoters (see Li et al., Nat. Biotech., 17:241-245 (1999)). Examples of promoters that are tissue-specific are known for liver (albumin, Miyatake et al., J. Virol., 71:5124-32 (1997); hepatitis B virus core promoter, Sandig et al., Gene Ther., 3:1002-9 (1996); alpha-fetoprotein (AFP), Arbuthnot et al., Hum. Gene Ther., 7:1503-14 (1996)), bone osteocalcin (Stein et al., Mol. Biol. Rep., 24:185-96 (1997)); bone sialoprotein (Chen et al., J. Bone Miner. Res., 11:654-64 (1996)), lymphocytes (CD2, Hansal et al., J. Immunol., 161:1063-8 (1998); immunoglobulin heavy chain; T cell receptor a chain), neuronal such as neuron-specific enolase (NSE) promoter (Andersen et al., Cell. Mol. Neurobiol., 13:503-15 (1993)), neurofilament light-chain gene (Piccioli et al., Proc. Natl. Acad. Sci. USA, 88:5611-5 (1991)), and the neuron-specific vgf gene (Piccioli et al., Neuron, 15:373-84 (1995)), among others. With respect to liver-specific promoters, examples include HLP, LP1, HCR-hAAT, ApoE-hAAT, and LSP. These promoters are described in more detail in the following references: HLP: McIntosh J. et al., Blood 2013 Apr. 25, 121(17):3335-44; LP1: Nathwani etal., Blood. 2006 April 1, 107(7): 2653-2661; HCR-hAAT: Miao et al., Mol Ther. 2000;1: 522-532; ApoE-hAAT: Okuyama et al., Human Gene Therapy, 7, 637-645(1996); and LSP: Wang et al., Proc Natl Acad Sci USA. 1999 March 30,96(7): 3906-3910. See, also Brown H. C. et al., Mol. Ther.: Meth. Clin. Dev. Vol. 9, pp:57-91, June 2018.

Examples of suitable activatable and constitutive promoters are known to those of skill in the art. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contains the rep and/or cap proteins under the control of inducible promoters. Still other stable host cells may be generated by one of skill in the art.

### Close Ended Linear Duplex Nucleic Acids

Closed linear DNA molecules typically comprise covalently closed ends also described as hairpin loops, where base-pairing between complementary DNA strands is not present. The hairpin loops join the ends of complementary DNA strands. Structures of this type typically form at the telomeric ends of chromosomes in order to protect against loss or damage of chromosomal DNA by sequestering the terminal nucleotides in a closed structure. In examples of closed linear DNA molecules described herein, hairpin loops flank complementary base-paired DNA strands, forming a closed linear (cl) DNA shaped structure. Closed linear DNA molecules include barbell shaped DNA.

One or more of the nucleic acids a)-c) i.e.: a) a nucleic acid sequence encoding helper proteins sufficient for rAAV replication; b) a nucleic acid sequence encoding rep and cap genes, and c) a close ended linear duplexed rAAV vector nucleic acid comprising at least one ITR and a heterologous transgene operably linked to one or more regulatory elements, may be present on close ended linear duplex nucleic acids. Such nucleic acids can be generated by a variety of known methods, including in vitro cell-free synthesis and in vivo methods.

In certain embodiments, the nucleic acid sequence containing one or more of a), b) or c) is an amplified linear open ended DNA, with blunt ends or with overhangs, and a synthesized hairpin molecule is ligated to one or both ends to form the closed ended linear DNA comprising one or more of the nucleic acids of a), b), or c). Unligated hairpins are purified away using means well known to those of skill in the art. The DNA may be amplified by PCR and ligated to double stranded form.

One method of generating the covalently closed ended linear duplex nucleic acids is by incorporation of protelomerase binding sites in a precursor molecule such that the protelomerase binding sites flank the nucleic acid of interest. The nucleic acid of interest can comprise one or more of a), b, and c), i.e. a), b) and c); any combination of a), b) and c); or only a), only b), or only c); and exposure of the molecule to protelomerase to thereby cleave and ligate the DNA at the site. Non-limiting examples of cell free in vitro synthesis are e.g. described in US 9,109,250; US 6,451,563; Nucleic Acids Res. 2015 Oct 15; 43(18): e120; US 9499847; 15/508,766; PCT/GB2017/052413; and Antisense & nucleic acid drug development 11:149-153 (2001); herein incorporated by reference in their entirety. The DNA from cell free in vitro synthesis is devoid of any prokaryotic DNA modifications.

A recombinant AAV vector genome can be designed having at least one of wild type ITR, synthetic ITR, or DD ITR, or a combination thereof, flanked by an imperfect palindromic structure containing protelomerase sites such as telRL. The template is used to produce closed linear double stranded nucleic acid vector when cleaved by a telomerase to form covalently closed ends. In one embodiment, the vector comprises two DD ITRs, an expression cassette, and flanked on each side of the DD ITRs is a telomerase target site, which can be cleaved by the telomerase to form covalently closes the ends. Closed linear DNA comprises half of protelomerase binding site.

In addition, a prokaryotic system can be used. In lysogenic bacteria, the bacteriophage N15 exists as a linear extrachromosomal DNA with covalently closed ends (see Rybchin VN, Svarchevsky AN (1999) The plasmid prophage N15: a linear DNA with covalently closed ends. Mol Microbiol 33:895-903). This DNA arises by a cleaving-joining reaction, which is exerted by a single enzyme, a protelomerase, for example, TelN (prokaryotic telomerase) [Deneke J, Ziegelin G, Lurz R, Lanka E (2000) The protelomerase of temperate Escherichia coli phage N15 has cleaving-joining activity. Proc Natl Acad Sci U S A 97:7721-7726]. A protelomerase such as TelN recognizes a target sequence in double-stranded DNA. The target site is an imperfect palindromic structure termed telRL, which is formed by the two halves telR and telL, corresponding to the covalently closed ends of the linear prophage. The enzyme cleaves both DNA strands and joins the resulting ends to form covalently closed hairpin structures. The resulting DNA molecule has two hairpin loops. TelN is able to linearize a recombinant plasmid harboring the telRL site [Deneke J, et al., (2000). Proc Natl Acad Sci U S A 97:7721-7726]. Therefore, one can employ this enzyme on a plasmid DNA for expression in higher organisms.

In certain embodiments, an in vivo cell system is used to produce_close ended linear duplex nucleic acids. The method comprises using a cell that expresses a protelomerase, such as TelN, or other protelomerase, wherein the protelomerase gene is under the control of a regulatable promoter. For example, an inducible promoter such as a small molecule regulated promoter or a temperature sensitive promoter, e.g. a heat shock promoter. After sufficient production of the AAV template DNA, or other nucleic acid of interest, or combination thereof, one can allow the protelomerase to be expressed which will excise the nucleic acid of interest, e.g. nucleic acid comprising one or more of a) helper, b) rep/cap, or c) AAV genome, from the template.

In certain embodiments, the in vivo cell system is used to produce a non-viral DNA vector construct for delivery of a predetermined nucleic acid sequence into a target cell for sustained expression. The non-viral DNA vector comprises, two DD-ITRs each comprising: an inverted terminal repeat having an A, A′, B, B′, C, C′ and D region; a D′ region; and wherein the D and D′ region are complementary palindromic sequences of about 5-20 nt in length, are positioned adjacent the A and A′ region; the predetermined nucleic acid sequence (e.g. a heterologous gene for expression); wherein the two DD-ITRs flank the nucleic acid in the context of covalently closed non-viral DNA and wherein the closed linear vector comprises a ½ protelomerase binding site on each end.

The TelN/telRL system described herein can be used to produce the closed linear DNA fragments either by linearizing a parental plasmid containing one telRL site or by excising the rAAV DNA fragment, or non-viral vector fragment, comprising a promoter, the gene of interest, a polyadenylation signal from the parental plasmid with two flanking ITRs, further having two telRL sites flanking the respective segment. In one embodiment, there is at least one double “D” ITR. The resulting linear covalently closed DNA molecules are functional in vivo.

The system comprises recombinant host cells. Suitable host cells for use in the present production system include microbial cells, for example, bacterial cells such as E. coli cells, and yeast cells such as S. cerevisiae. Mammalian host cells may also be used including Chinese hamster ovary (CHO) cell for example of K1 lineage (ATCC CCL 61) including the Pro5 variant (ATCC CRL 1281); the fibroblast-like cells derived from SV40-transformed African Green monkey kidney of the CV-1 lineage (ATCC CCL 70), of the COS-1 lineage (ATCC CRL 1650) and of the COS-7 lineage (ATCC CRL 1651; murine L-cells, murine 3T3 cells (ATCC CRL 1658), murine C127 cells, human embryonic kidney cells of the 293 lineage (ATCC CRL 1573), human carcinoma cells including those of the HeLa lineage (ATCC CCL 2), and neuroblastoma cells of the lines IMR-32 (ATCC CCL 127), SK-N-MC (ATCC HTB 10) and SK-N-SH (ATCC HTB 11).

The host cell is designed to encode at least one recombinase. The host cell may also be designed to encode two or multiple recombinases. The term “recombinase” refers to an enzyme that catalyzes DNA exchange at a specific target site, for example, a palindromic sequence, by excision/insertion, inversion, translocation and exchange. Examples of suitable recombinases for use in the present system include, but are not limited to, TelN, Tel, Tel (gp26 K02 phage) Cre, Flp, phiC31, Int and other lambdoid phage integrases, e.g. phi 80, HK022 and HP1 recombinases. The target sequences for each of these recombinases are, respectively: the telRL site:

TATCAGCACACAATTGCCCATTATACGCGCGTATAATGGACTATTGTGTGCTG
A (SEQ ID NO: 15);
the pal site: ACCTATTTCAGCATACTACGCGCGTAGTATGCTGAAATAGGT
(SEQ ID NO: 16);
the φK02 telRL site: CCATTATACGCGCGTATAATGG (SEQ ID NO: 17);
the loxP site: TAACTTCGTATAGCATACATTATACGAAGTTAT (SEQ ID NO:
18);
the FRT site: GAAGTTCCTATTCTCTAGAAAGTATAGGAACTTC (SEQ ID NO:
19)
the phiC31 attP site:
CCCAGGTCAGAAGCGGTTTTCGGGAGTAGTGCCCCAACTGGGGTAACCTTTGA
GTTCTCTCAGTT GGGGGCGTAGGGTCGCCGACAYGACACAAGGGGTT (SEQ ID
NO: 20); and
the λ attP site:
TGATAGTGACCTGTTCGTTGCAACACATTGATGAGCAATGCTTTTTTATAATGC
CAACTTTGTACAA AAAAGCTGAACGAGAAACGTAAAATGATATAAA (SEQ ID
NO: 21).

Expression of the recombinase is under the control of any regulated or inducible promoter, i.e. a promoter which is activated under a particular physical or chemical condition or stimulus. Examples of suitable promoters include thermally-regulated promoters such as the λpL promoter, the IPTG regulated lac promoter, the glucose regulated ara promoter, the T7 polymerase regulated promoter, cold-shock inducible cspA promoter, pH inducible promoters, or combinations thereof, such as tac (T7 and lac) dual regulated promoter.

Alternate methods of generating covalently closed end linear DNA that lack bacterial sequences are known in the art e.g., by formation of mini-circle DNA from plasmids (e.g. as described in U.S. Pat. 8,828,726, and U.S. Pat. 7,897,380, the contents of each of which are incorporated by reference in their entirety). For example, one method of cell-free synthesis combines the use of two enzymes - Phi29 DNA polymerase and a protelomerase, and generates high fidelity, covalently closed, linear DNA constructs. The constructs contain no antibiotic resistance markers, and therefore eliminate the packaging of these sequences. The process can amplify AAV genome DNA in a 2-week process at commercial scale and maintain the ITR sequences required for virus production.

Phi29 DNA polymerase is used to amplify double-stranded DNA by rolling circle amplification, and a protelomerase to generate covalently closed linear DNA, which coupled with a streamlined purification process, results in a pure DNA product containing just the sequence of interest. Phi29 DNA polymerase has high fidelity (1 × 10⁶-1 × 10⁷) and high processivity (approximately 70 kbp). These features make this polymerase particularly suitable for the large-scale production of GMP DNA. Protelomerases (also known as telomere resolvases) catalyze the formation of covalently closed hairpin ends on linear DNA and have been identified in some phages, bacterial plasmids and bacterial chromosomes. A pair of protelomerases recognizes inverted palindromic DNA recognition sequences and catalyzes strand breakage, strand exchange and DNA ligation to generate closed linear hairpin ends. The formation of these closed ended structures makes the DNA resistant to exonuclease activity, allowing for simple purification and can improve stability and duration of expression.

### Protelomerase Binding Sites

In one embodiment, the DNA construct comprises a protelomerase binding site and the covalently closed ends are formed by protelomorase enzyme activity (e.g., in vitro). Protelomerase binding sites and corresponding protelomerases for use in the invention are provided in U.S. Pat. No. 9,499,847, the contents of which are incorporated herein by reference in their entirety. A protelomerase target sequence as used in the invention preferably comprises a double stranded palindromic (perfect inverted repeat) sequence of at least 14 base pairs in length. Preferred perfect inverted repeat sequences include the sequences of SEQ ID NOs: 1 to 6 and variants thereof. SEQ ID NO: 1 (NCATNNTANNCGNNTANNATGN) is a 22 base consensus sequence for a mesophilic bacteriophage perfect inverted repeat. Base pairs of the perfect inverted repeat are conserved at certain positions between different bacteriophages, while flexibility in sequence is possible at other positions. Thus, SEQ ID NO: 1 is a minimum consensus sequence for a perfect inverted repeat sequence for use with a bacteriophage protelomerase in the process of the present invention.

Within the consensus defined by SEQ ID NO: 1, SEQ ID NO: 2 (CCATTATACGCGCGTATAATGG) is a perfect inverted repeat sequence for use with E. coli phage N15, and Klebsiella phage Phi KO2 protelomerases. Also within the consensus defined by SEQ ID NO: 1, SEQ ID NOs: 3 to 5: SEQ ID NO: 3 (GCATACTACGCGCGTAGTATGC), SEQ ID NO: 4 (CCATACTATACGTATAGTATGG), SEQ ID NO: 5 (GCATACTATACGTATAGTATGC), are particularly preferred perfect inverted repeat sequences for use respectively with protelomerases from Yersinia phage PY54, Halomonas phage phiHAP-1, and Vibrio phage VP882. SEQ ID NO: 6 (ATTATATATATAAT) is a particularly preferred perfect inverted repeat sequence for use with a Borrelia burgdorferi protelomerase. This perfect inverted repeat sequence is from a linear covalently closed plasmid, lpB31.16 comprised in Borrelia burgdorferi. This 14 base sequence is shorter than the 22 bp consensus perfect inverted repeat for bacteriophages (SEQ ID NO: 1), indicating that bacterial protelomerases may differ in specific target sequence requirements to bacteriophage protelomerases. However, all protelomerase target sequences share the common structural motif of a perfect inverted repeat.

The perfect inverted repeat sequence may be greater than 22 bp in length depending on the requirements of the specific protelomerase used in the process of the invention. Thus, in some embodiments, the perfect inverted repeat may be at least 30, at least 40, at least 60, at least 80 or at least 100 base pairs in length. Examples of such perfect inverted repeat sequences include SEQ ID NOs: 7 to 9 and variants thereof. SEQ ID NO: 7 (GGCATAC TATACGTATAGTATGCC); SEQ ID NO: 8 (ACCTATTTCAGCATACTACGCGCGTAGTATGCTGAAATAGGT); SEQ ID NO: 9 (CCTATATTGGGCCACCTATGTATG-CACAGTTCGCCCATACTATACGTATAGTATGGGCGAACTGTGCATACATAGGT GGCCCAATATAGG). SEQ ID NOs: 7 to 9 and variants thereof are particularly preferred for use respectively with protelomerases from Vibrio phage VP882, Yersinia phage PY54 and Halomonas phage phi HAP-1.

The perfect inverted repeat may be flanked by additional inverted repeat sequences. The flanking inverted repeats may be perfect or imperfect repeats i.e may be completely symmetrical or partially symmetrical. The flanking inverted repeats may be contiguous with or non-contiguous with the central palindrome. The protelomerase target sequence may comprise an imperfect inverted repeat sequence which comprises a perfect inverted repeat sequence of at least 14 base pairs in length. An example is SEQ ID NO: 14. The imperfect inverted repeat sequence may comprise a perfect inverted repeat sequence of at least 22 base pairs in length. An example is SEQ ID NO: 10.

In certain embodiments, the protelomerase target sequences comprise the sequences

of SEQ ID NOs: 10 to 14 or variants thereof. SEQ ID NO: 10:
(TATCAGCACACAATTGCCCATTATACG-CGCGTATAATGGACTATTG
TGTGCTGATA); SEQ ID NO: 11
(ATGCGCGCATCCATTATACGCGCGTATAATGGCGATAATACA); SEQ ID NO: 12
(TAGTCACCTATTTCAGCATACTACGCGCGTAGTATGCTGAAATAGG TTACTG);
SEQ ID NO: 13:
(GGGATCCCGTTCCATACATACATGTATCCATGTGGCATACTATACG
TATAGTATGCCGATGTTACATATGGTATCATTCGGGATCCCGTT); SEQ ID NO:
14 (TACTAAATAAATATTATATATATAATTTTTTATTAGTA).

The sequences of SEQ ID NOs: 10 to 14 comprise perfect inverted repeat sequences as described above, and additionally comprise flanking sequences from the relevant organisms. A protelomerase target sequence comprising the sequence of SEQ ID NO: 10 or a variant thereof is preferred for use in combination with E. coli N15 TelN protelomerase and variants thereof. A protelomerase target sequence comprising the sequence of SEQ ID NO: 11 or a variant thereof is preferred for use in combination with Klebsiella phage Phi K02 protelomerase and variants thereof. A protelomerase target sequence comprising the sequence of SEQ ID NO: 12 or a variant thereof is preferred for use in combination with Yersinia phage PY54 protelomerase and variants thereof. A protelomerase target sequence comprising the sequence of SEQ ID NO: 13 or a variant thereof is preferred for use in combination with Vibrio phage VP882 protelomerase and variants thereof. A protelomerase target sequence comprising the sequence of SEQ ID NO: 14 or a variant thereof is preferred for use in combination with a Borrelia burgdorferi protelomerase.

Variants of any of the palindrome or protelomerase target sequences described above include homologues or mutants thereof. Mutants include truncations, substitutions or deletions with respect to the native sequence. A variant sequence is any sequence whose presence in the DNA template allows for its conversion into a closed linear DNA by the enzymatic activity of protelomerase. This can readily be determined by use of an appropriate assay for the formation of closed linear DNA. Any suitable assay described in the art may be used. An example of a suitable assay is described in Deneke et al., PNAS (2000) 97, 7721-7726. In certain embodiments, the variant allows for protelomerase binding and activity that is comparable to that observed with the native sequence. Examples of preferred variants of palindrome sequences described herein include truncated palindrome sequences that preserve the perfect repeat structure, and remain capable of allowing for formation of closed linear DNA. However, variant protelomerase target sequences may be modified such that they no longer preserve a perfect palindrome, provided that they are able to act as substrates for protelomerase activity.

It should be understood that the skilled person would readily be able to identify suitable protelomerase target sequences for use in the invention on the basis of the structural principles outlined above. Candidate protelomerase target sequences can be screened for their ability to promote formation of closed linear DNA using the assays described above.

### Generation of Covalently Closed Linear DNA Construct

The covalently closed vectors described herein may be generated in vitro or in vivo. The vectors are covalently closed linear double stranded vectors capable of expressing transgene in a target cell. One example of an in vitro process for the production of a closed linear expression cassette DNA, e.g. containing the ITRs described herein, comprises a) contacting a DNA template comprising at least one expression cassette flanked on either side by a protelomerase target sequence with at least one DNA polymerase in the presence of one or more primers under conditions promoting amplification of said template; and b) contacting amplified DNA produced in a) with at least one, protelomerase under conditions promoting formation of a closed linear expression cassette DNA. The closed linear expression cassette DNA product may comprise, consist or consist essentially of a eukaryotic promoter operably linked to a coding sequence of interest, and optionally a eukaryotic transcription termination sequence. The closed linear expression cassette DNA product may additionally lack one or more bacterial or vector sequences, typically selected from the group consisting of: (i) bacterial origins of replication; (ii) bacterial selection markers (typically antibiotic resistance genes) and (iii) unmethylated CpG motifs.

As outlined above, any DNA template comprising at least one protelomerase target sequence may be amplified according to the process of the invention. Thus, although production of therapeutic DNA molecules, e.g. for DNA vaccines or other therapeutic proteins and nucleic acid is preferred, the process of the invention may be used to produce any type of closed linear DNA. The DNA template may be a double stranded (ds) or a single stranded (ss) DNA. A double stranded DNA template may be an open circular double stranded DNA, a closed circular double stranded DNA, an open linear double stranded DNA or a closed linear double stranded DNA. Preferably, the template is a closed circular double stranded DNA Closed circular dsDNA templates are particularly preferred for use with RCA (rolling circle amplification) DNA polymerases. A circular dsDNA template may be in the form of a plasmid or other vector typically used to house a gene for bacterial propagation. Thus, the process of the invention may be used to amplify any commercially available plasmid or other vector, such as a commercially available DNA medicine, and then convert the amplified vector DNA into closed linear DNA.

An open circular dsDNA may be used as a template where the DNA polymerase is a strand displacement polymerase which can initiate amplification from at a nicked DNA strand. In this embodiment, the template may be previously incubated with one or more enzymes which nick a DNA strand in the template at one or more sites. A closed linear dsDNA may also be used as a template. The closed linear dsDNA template (starting material) may be identical to the closed linear DNA product. Where a closed linear DNA is used as a template, it may be incubated under denaturing conditions to form a single stranded circular DNA before or during conditions promoting amplification of the template DNA. In one embodiment, the close ended linear duplex DNA is produced in eukaryotic cells for example insect cells as described in PCT publications WO 2019032102 and WO 2019169233. In one embodiment, the DNA is not produced in eukaryotic cells and DNA lacks eukaryotic sequences. In one embodiment, the close ended liner duplex DNA vectors are produced as described in PCT publication WO 2019143885.

As outlined above, the DNA template typically comprises an expression cassette as described above, i.e., comprising, consisting or consisting essentially of a eukaryotic promoter operably linked to a sequence encoding a protein of interest, and optionally a eukaryotic transcription termination sequence. Optionally the expression cassette may be a minimal expression cassette as defined above, i.e. lacking one or more bacterial or vector sequences, typically selected from the group consisting of: (i) bacterial origins of replication; (ii) bacterial selection markers (typically antibiotic resistance genes) and (iii) unmethylated CpG motifs.

### Cell Culture Medium

As used herein, the terms “cell culture medium” and “culture medium” refer to a nutrient solution used for growing cells in vitro that typically provides at least one component from one or more of the following categories: 1) an energy source, usually in the form of a carbohydrate such as, for example, glucose; 2) one or more of all essential amino acids, and usually the basic set of twenty amino acids; 3) vitamins and/or other organic compounds required at low concentrations; 4) free fatty acids; and 5) trace elements, where trace elements are defined as inorganic compounds or naturally occurring elements that are typically required at very low concentrations, usually in the micromolar range. The nutrient solution may optionally be supplemented with additional components to optimize growth and/or transfection of cells.

The cell culture within the present invention is prepared in a medium suitable for the particular host cell being cultured. Suitable cell culture media that may be used for culturing a particular cell type would be apparent to one of ordinary skill in the art. Exemplary commercially available media include, for example, Ham’s F 10 (SIGMA), Minimal Essential Medium (MEM, SIGMA), RPMT-1640 (SIGMA), Dulbecco’s Modified Eagle’s Medium (DMEM, SIGMA); Iscove modified Dulbecco medium (Gibco) containing 10% fetal bovine serum (see, Xiao et al, Production of High-Titer Recombinant Adeno-Associated Virus Vectors in the Absence of Helper Adenovirus, J Virol, 72: 2224- 2232 (1998)), and DMEM/F 12 (Life Technologies). Any of these or other suitable media may be supplemented as necessary with hormones and/or other growth factors (such as but not limited to insulin, transferrin, or epidermal growth factor), salts (such as sodium chloride, calcium, magnesium, and phosphate), buffers (such as HEPES), nucleosides (such as adenosine and thymidine), antibiotics (such as puromycin, neomycin, hygromycin, blasticidin, or Gentamycin™), trace elements (defined as inorganic compounds usually present at final concentrations in the micromolar range) lipids (such as linoleic or other fatty acids) and their suitable carriers, and glucose or an equivalent energy source, and/or modified as described herein to facilitate production of recombinant glycoproteins having low-mannose content.

Depending upon the requirements of the particular cell line used or method, culture medium can contain a serum additive such as Fetal Bovine Serum, or a serum replacement. Examples of serum-replacements (for serum-free growth of cells) are TCH™, TM-235™, and TCH™; these products are available commercially from Celox (St. Paul, Minn.), and KOSR (knockout (KO) serum replacement; Life Technologies).

In certain embodiments of any one of the aspects, host cells can be grown in serum-free, protein-free, growth factor-free, and/or peptone-free media. The term “serum-free” as applied to media in general includes any mammalian cell culture medium that does not contain serum, such as fetal bovine serum (FBS). The term “growth- factor free” as applied to media includes any medium to which no exogenous growth factor (e.g., insulin, IGF-1) has been added. The term “peptone-free” as applied to media includes any medium to which no exogenous protein hydrolysates have been added such as, for example, animal and/or plant protein hydrolysates.

In some embodiments of any one of the aspects, the cell culture medium is serum-free. By “serum-free”, it is understood that the concentration of serum in the medium is preferably less than 0.1% (v/v) serum and more preferably less than 0.01% (v/v) serum By “essentially serum-free” is meant that less than about 2% (v/v) serum is present, more preferably less than about 1% serum is present, still more preferably less than about 0.5% (v/v) serum is present, yet still more preferably less than about 0.1% (v/v) serum is present. When defined medium that is serum-free is used, the medium is usually enriched for particular amino acids, vitamins and/or trace elements (see, for example, U.S. Pat. No. 5, 122,469 to Mather et al, and U.S. Pat. No. 5,633, 162 to Keen et al).

“Culturing” or “incubating” (used interchangeably with respect to the growth, transformation and/or maintenance of host cells or host cell lines) is under conditions of sterility, temperature, pH, atmospheric gas content (e.g., oxygen, carbon dioxide, dinitrogen), humidity, culture container, culture volume, passaging, motion, and other parameters suitable for the intended purpose and conventionally known in the art of mammalian cell culture.

In certain embodiments, the culture medium comprises an amino acid at a concentration of from about 1 mM to about 100 mM. For example, the culture medium comprises an amino acid at a concentration of from about 1 mM to about 20 mM, e.g., from about 5 mM to about 15 mM. In certain embodiments, the culture medium comprises an amino acid at a concentration of from about 7.5 mM to about 12.5 mM. For example, the culture medium comprises an amino acid at a concentration of about 10 mM.

In certain embodiments, the culture medium comprises L-glutamine or a dipeptide comprising L-glutamine. An exemplary dipeptide comprising L-glutamine is L-alanyl-L-glutamine (e.g., GLUTAMAX™). Generally, the culture medium comprises L-glutamine or a dipeptide comprising L-glutamine at a concentration of from about 1 mM to about 100 mM. For example, the culture medium comprises L-glutamine or a dipeptide comprising L-glutamine at a concentration of from about 1 mM to about 20 mM, e.g., from about 5 mM to about 15 mM. In certain embodiments, the culture medium comprises L-glutamine or a dipeptide comprising L-glutamine at a concentration of from about 7.5 mM to about 12.5 mM. For example, the culture medium comprises L-glutamine or a dipeptide comprising L-glutamine at a concentration of about 10 mM.

In certain embodiments, the culture medium can also comprise a nonionic surfactant polyol or detergent. Exemplary non-ionic detergents include, but are not limited to, polysorbates such as polysorbate 20 (TWEEN 20), polysorbate 28, polysorbate 40, polysorbate 60, polysorbate 65, polysorbate 80, polysorbate 81, and polysorbate 85; poloxamers such as poloxamer 188, poloxamer 407; polyethylene polypropylene glycol; or polyethylene glycol (PEG). In some embodiments of any one of the aspects, the nonionic surfactant polyol or detergent is a poloxomer. Exemplary poloxomers include, but are not limited to, Poloxamer 188 (P188), Pluronic® F127, Pluronic® F38, Pluronic® F68, Pluronic® F87, Pluronic® F108, Pluronic® 10R5, Pluronic® 17R2, Pluronic® 17R4, Pluronic® 25R2, Pluronic® 25R4, Pluronic® 31R1, Pluronic® F108 Cast Solid Surfacta, Pluronic® F108 NF, Pluronic® F108 Pastille, Pluronic® F108NF Prill Poloxamer 338, Pluronic® F127 NF, Pluronic® F127 NF 500 BHT Prill, Pluronic® F127 NF Prill Poloxamer 407, Pluronic® F38 Pastille, Pluronic® F68 LF Pastille, Pluronic® F68 NF, Pluronic® F68 NF Prill, Pluronic® F68 Pastille, Pluronic® F77, Pluronic® F77 Micropastille, Pluronic® F87 NF, Pluronic® F87 NF Prill Poloxamer 237, Pluronic® F 88, Pluronic® F 88 Pastille, Pluronic® F 98, Pluronic® FT L 61, Pluronic® L10, Pluronic® L101, Pluronic® L121, Pluronic® L31, Pluronic®L35, Pluronic® L43, Pluronic®L61, Pluronic® L62, Pluronic® L62 LF, Pluronic® L62D, Pluronic® L64, Pluronic® L81, Pluronic® L92, Pluronic® L44 NF INH surfactant Poloxamer 124, Pluronic® N3, Pluronic® P103, Pluronic® P104, Pluronic® P105, Pluronic® P123 Surfactant, Pluronic® P65, Pluronic® P84, Pluronic® P85, and the like.

The amount of the nonionic surfactant polyol or detergent in the culture medium can be at least about 0.01%, 0.015%, 0.02%, 0.025%, 0.03%, 0.035%, 0.04%, 0.045%, 0.05% (w/w, w/v or v/v) or more. For example, the amount of the nonionic surfactant polyol or detergent in the culture medium can range from about 0.001% to about 1% (weight/volume). For example, the culture medium comprises the nonionic surfactant polyol or detergent at a concentration of from about 0.01% to about 0.5%, from about 0.015% to about 0.45%, from about 0.02% to about 0.4%, or from about 0.025% to about 0.35%. 0.1%. For example, the culture medium comprises the nonionic surfactant polyol or detergent at a concentration of about 0.01%, about 0.015%, about 0.02%, about 0.025%, about 0.03%, about 0.035%, about 0.04%, about 0.045%, or about 0.05%.

In certain embodiments, the culture medium comprises an anti-foaming agent. The term “anti-foaming agent” refers to a chemical that, when added to a fluid, can substantially reduce the surface activity of the fluid thereby substantially preventing the fluid from foaming. Exemplary anti-foaming agents amenable to the present invention include, but are not limited to, high molecular weight silicones and other materials well known in the art for such use.

The amount of the anti-foaming agent in the culture medium can be at least about 0.01%, 0.015%, 0.02%, 0.025%, 0.03%, 0.035%, 0.04%, 0.045%, 0.05% (w/w, w/v or v/v) or more. For example, the amount of the anti-foaming agent in the culture medium can range from about 0.001% to about 1% (weight/volume). For example, the culture medium comprises the anti-foaming agent at a concentration of from about 0.01% to about 0.5%, from about 0.015% to about 0.45%, from about 0.02% to about 0.4%, or from about 0.025% to about 0.35%. 0.1%. For example, the culture medium comprises the anti-foaming agent at a concentration of about 0.01%, about 0.015%, about 0.02%, about 0.025%, about 0.03%, about 0.035%, about 0.04%, about 0.045%, or about 0.05%.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

### Lysis of Host Cells

In certain embodiments of any one of the aspects, the method comprises lysing the transfected host cell. Methods for lysing host cells in a cell culture are well known in the art. For example, a non-ionic surfactant can be added to the cell culture or cell culture supernatant. Generally, the non-ionic surfactant is added to the cell culture to a final concentration of at least about 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1% (w/v, w/w or v/v) or higher. For example, the non-ionic surfactant is added to the cell culture to a final concentration of from about 0.05% to about 1%, from about 0.1% to about 0.95%, from about 0.15% to about 0.9%, from about 0.2% to about 0.85%, from about 0.25% to about 0.8%, from about 0.3% to about 0.75%, from about 0.35% to about 0.65% from about 0.4% to about 0.6% or from 0.45% to about 0.55%. In some embodiments, the non-ionic surfactant is added to the cell culture to a final concentration of about 0.05%, 0.1%., about 0.15%, about 0.2%, about 0.25%, about 0.3%, about 0.35%, about 0.4%, about 0.45%, about 0.5%, about 0.55%, about 0.6%, about 0.65%, about.7%, about 0.75%, about 0.8%, about 0.85%, about 0.9%, about 0.95%, or about 1%. For example, the non-ionic surfactant can be added to the cell culture to a final concentration of about 0.5%.

Generally, the non-ionic surfactant is allowed to mix with the cell culture for a sufficient period of time to lyse host cells present in the cell culture or cell culture supernatant. For example, the non-ionic surfactant is mixed with the cell culture for a period of from about 15 minutes to about 2 hours. In some embodiments, the non-ionic surfactant is mixed with the cell culture for a period of from about 30 minutes to about 60 minutes.

The mixing can be at ambient temperature or an elevated temperature. For example, the mixing with the non-ionic surfactant can be at a temperature from about 15° C. to about 37° C. In some embodiments, the mixing with the non-ionic surfactant can be at a temperature of about 18° C., 19° C., 20° C., 21° C., 22° C., 23° C., 24° C., 25° C., 26° C., 27° C., 28° C., 28° C., 30° C., 31° C., 32° C., 33° C., 34° C., 35° C., 36° C., or 37° C.

It is noted that, any desired non-ionic surfactant can be used for lysing the transfected host cells. Exemplary non-ionic surfactants and classes of non-ionic surfactants for lysing the transfected host cells can include polyarylphenol polyethoxy ethers; polyalkylphenol polyethoxy ethers; polyglycol ether derivatives of saturated fatty acids; polyglycol ether derivatives of unsaturated fatty acids; polyglycol ether derivatives of aliphatic alcohols; polyglycol ether derivatives of cycloaliphatic alcohols; fatty acid esters of polyoxyethylene sorbitan; alkoxylated vegetable oils; alkoxylated acetylenic dials; polyalkoxylated alkylphenols; fatty acid alkoxylates; sorbitan alkoxylates; sorbitol esters; C₈ to C₂₂ alkyl or alkenyl polyglycosides; polyalkoxy styrylaryl ethers; alkylamine oxides; block copolymer ethers; polyalkoxylated fatty glyceride; polyalkylene glycol ethers; linear aliphatic or aromatic polyesters; organo silicones; polyaryl phenols; sorbitol ester alkoxylates; and mono- and diesters of ethylene glycol and mixtures thereof, ethoxylated tristyrylphenol; ethoxylated fatty alcohol; ethoxylated lauryl alcohol; ethoxylated castor oil; and ethoxylated nonylphenol; alkoxylated alcohols, amines or acids. In some embodiments of any one of the aspects, the non-ionic surfactant for lysing the host cells is selected from the group consisting of polyoxyethylene fatty alcohol ethers, polyoxyethylene alkylphenyl ethers, polyoxyethylene-polyoxypropylene block copolymers, alkylglucosides, alkylphenol ethoxylates, preferably polysorbates, polyoxyethylene alkyl phenyl ethers, and any combinations thereof.

Specific exemplary non-ionic surfactants for lysing the transfected host cells include, but are not limited to, ECOSURF EH-9, polysorbates (such as polysorbate 20 (TWEEN 20), polysorbate 28, polysorbate 40, polysorbate 60, polysorbate 65, polysorbate 80, polysorbate 81, and polysorbate 85), ECOSURF EH-14, TWEEN 60 nonionic detergent, PPG-PEG-PPG Pluronic 10R5, Polyoxyethylene (18) tridecyl ether, Polyoxyethylene (12) tridecyl ether, MERPOL SH surfactant,MERPOL OJ surfactant, MERPOL HCS surfactant, IGEPAL CO-720, IGEPAL CO-630, IGEPAL CA-720, Brij S20, BrijS10, Brij 010, Brij C10, BRIJ 020, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, and TERGITOLNP-13 and any combinations thereof. In some embodiments, the non-ionic surfactant for lysing the transfected host cells is not Triton X-100.

In some embodiments, a zwitterionic surfactant can be added to the cell culture for lysing the transfected host cell. Exemplary zwitterionic surfactants include, but are not limited to, sulfonates, such as CHAPS (3-[(3-Cholamidopropyl)dimethylammonio]-1-propanesulfonate), CHAPSO (3-{(3-cholamidopropyl)dimethylammonio}-2-hydroxy-1-propane-sulfonate), 3-(decyldimethylammonio)propanesulfonate, 3-(dodecyldimethylammonio) propanesulfonate, 3-(N,N-dimethylmyristylammonio)propanesulfonate, 3-(N,N-dimethyl octadecylammonio)propane sulfonate, 3-(N,N-dimethyloctylammonio)propanesulfonate, and 3-(N,N-dimethyl palmityl ammonio)propanesulfonate; sultaines, such as cocamidopropyl hydroxysultaine; betaines, e.g., cocamidopropyl betaine; and phosphates, such as lecithin.

In some embodiments, the surfactant, e.g., the zwitterionic surfactant can be an amine oxide surfactant. For example, an amine oxide surfactant can be added to the cell culture for lysing the host cell. An amine oxide surfactant that can be used in methods described herein can be a trialkyl amine N-oxide, e.g., an amine oxide of formula R¹R²R³NO, wherein R¹ is a substituted or unsubstituted alkyl or alkenyl containing from about 8 to about 30 carbon atoms; and R² and R³ are independently substituted or unsubstituted alkyl or alkenyl groups containing from about 1 to about 18 carbon atoms. Non limiting examples of trialkyl amine N-oxide and trialkyl amine N-oxide surfactants of use are described in WO1998055581, which is incorporated herein by reference in its entirety.

The lysate may comprise impurities, e.g., host cell DNA (hcDNA). Therefore, the method can comprise a post-lysis step of removing or reducing amount of impurities, e.g., hcDNA from the lysate prior to isolating/purifying the rAAV. Methods and compositions for reducing the amount of host cell DNA in cell cultures or cell culture supernatants are well known in the art. For example, a cationic amine or nuclease can be added to the lysate.

In some embodiments, the post-lysis step comprises adding a selective precipitation agent to reduce or remove impurities such as hcDNA from the lysate. As used herein, a “selective precipitation agent” refers to any agent, compound or such which, when added to a preparation comprising a population of recombinant virus particles and contaminating nucleic acid molecules, will affect the selective precipitation of at least a substantial amount of contaminating nucleic acid molecules away from the recombinant virus particles. Exemplary agents for adding to the lysate in the post-lysis step include, but are not limited to cetyl trimethylammonium bromide, cetylpyridinium chloride, benzethonium chloride, tetradecyltrimethyl-ammonium chloride, polyethylene imine and combinations thereof.

In some embodiments, a nuclease, e.g., an endonuclease is added to the lysate for reducing or removing impurities such as hcDNA. Exemplary endonucleases include endonucleases derived from both Prokaryotes and Eukaryotes. In some embodiments, the nuclease is BENZONASE® or a salt active nuclease (SAN).

Generally, the nuclease is added to the lysate to a final concentration of at least about 0.05%, 0.1%., 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1% (w/v, w/w or v/v) or higher. For example, the nuclease is added to the lysate to a final concentration of from about 0.05% to about 1%, from about 0.1% to about 0.95%, from about 0.15% to about 0.9%, from about 0.2% to about 0.85%, from about 0.25% to about 0.8%, from about 0.3% to about 0.75%, from about 0.35% to about 0.65% from about 0.4% to about 0.6%, from 0.45% to about 0.55% from about 0.05% to about 0.4%, or from about 0.2% to about 0.4%. In some embodiments, the nuclease is added to the lysate to a final concentration of about 0.05%, 0.1%., about 0.15%, about 0.2%, about 0.25%, about 0.3%, about 0.35%, about 0.4%, about 0.45%, about 0.5%, about 0.55%, about 0.6%, about 0.65%, about.7%, about 0.75%, about 0.8%, about 0.85%, about 0.9%, about 0.95%, or about 1%. For example, the nuclease can be added to the lysate to a final concentration of about 0.2%. In some embodiments, the nuclease can be added to the lysate to a final concentration of about 0.05% to about 0.4%.

Generally, the agent or nuclease is allowed to mix with the lysate for a period of about 15, 20, 30, 35, 40, 45, 50, 55 minutes or longer. In some embodiments, the agent or nuclease is allowed to mix with the lysate for a period of from about 10 minutes to about 4 hours. For example, the agent or nuclease is mixed with the lysate for a period of from about 15 minutes to about 3 hours. In some embodiments, the agent or nuclease is mixed with the lysate for a period of from about 30 minutes to about 120 minutes. For example, the agent or nuclease is mixed with the lysate for a period of about 30 minutes.

In some embodiment, the method comprises a step of clarifying the lysate. For example, the method comprises a step of clarifying the lysate by depth filtration to produce a clarified composition.

### Isolation/Purification of rAAV

Several methods for isolating/purifying rAAV from a lysate from a host cell line are known in the art. Such, methods include, but are not limited to density gradient, tangential flow filtration, affinity chromatography, size exclusion chromatography, cation exchange chromatography, anion exchange chromatography, hydroxylapatite chromatography, hydrophobic interaction chromatography, and various combinations thereof. Exemplary methods for isolating/purifying the rAAV from a host cell lysate are described, for example, in U.S. Pat. No. 6,592,123; U.S. Pat. No. 9,862,936; Int. Pat. Pub. No. WO2019/241535; Int. Pat. Pub. No. W02005/035743; and Int. Pat. Pub. No. WO2019/212921, contents of all of which are incorporated herein by reference in their entireties.

Aspects of the invention can be described by the following numbered Embodiments 1-103: Embodiment 1: A method of producing a recombinant adeno-associated virus (rAAV) lacking prokaryotic sequences comprising: (i) optionally, culturing a human embryonic cell line in suspension; (ii) transfecting the human embryonic cell line with a) a nucleic acid sequence encoding helper proteins sufficient for rAAV replication; b) a nucleic acid sequence encoding AAV rep and AAV cap genes, and c) a close ended linear duplexed rAAV vector nucleic acid comprising at least one inverted terminal repeat (ITR) sequence and a heterologous transgene operably linked to one or more regulatory elements; (iii) incubating the transfected human cell line for between about 40 to 400 hours; and (iv) lysing the transfected human cell line and purifying the nucleic acid sequences encoding the rAAV, thereby producing the rAAV.

Embodiment 2: The method of claim 1, wherein cells of said cell line are transfected in suspension.

Embodiment 3: The method of any one of claims 1-2, wherein the human embryonic cell line is suspension-adapted, serum-free cell line derived from a human embryonic kidney cell line.

Embodiment 4: The method of any one of claims 1-3, wherein the AAV rep and AAV cap genes are from different serotypes.

Embodiment 5: The method of any one of claims 1-3, wherein the AAV rep and AAV cap genes are from same serotypes.

Embodiment 6: The method of any one of claims 1-5, wherein the AAV rep gene is an AAV2 rep gene and the AAV cap gene is an AAV8 cap gene.

Embodiment 7: The method of any one of claims 1-6, wherein the AAV ITR and the AAV cap genes are from different serotypes.

Embodiment 8: The method of any one of claims 1-6, wherein the AAV ITR and the AAV cap genes are from same serotype.

Embodiment 9: The method of any one of claims 1-8, wherein the AAV inverted terminal repeat (ITR) sequences are adeno-associated virus 2 inverted terminal repeat (ITR) sequences.

Embodiment 10: The method of any one of claims 1-9, wherein the AAV ITR sequence is from AAV 2 serotype or serotypes selected from the group consisting of AAV1, 3a, 3b, 4, 5, 6, 7, 8, 9, 10, 11, and 13.

Embodiment 11: The method of any one of claims 1-10, wherein the AAV ITR sequence is synthetic.

Embodiment 12: The method of any one of claims 1-11, wherein the total amount of nucleic acid transfected from a), b), and c) per 1 × 10⁶ cells is less than 2 µg, optionally, the total amount of nucleic acid transfected from a), b), and c) per 1 × 10⁶ cells is less than 1 µg.

The Embodiment 13: The method of any one of claims 1-12, wherein the ratio of a):b):c) is about 0.5-1.75: about 0.75-2.25: about 0.5-1.75 (weight:weight:weight), optionally, the ratio of a):b):c) is about 1:about 1-1.6: about 1 (weight:weight:weight).

Embodiment 14: The method of any one of claims 1-13, wherein a), b) and c) are transfected using a transfection composition comprising a), b) and c), and a stable cationic polymer, wherein the ratio of stable cationic polymer to total amount of nucleic acid from a), b) and c), is from about 1:1 to about 3:1 (weight/weight), optionally, the ratio of stable cationic polymer to total amount of nucleic acid from a), b) and c), is about 1.5:1. Embodiment 15: The method of any one of claims 1-14, wherein each of a), b) and c) are provided on one or more close ended linear duplexed nucleic acid molecules.

Embodiment 16: The method of any one of claims 1-15, wherein the transfected nucleic acids a), b), and c) are synthetic nucleic acids and devoid of eukaryotic and prokaryotic cellular modifications of DNA.

Embodiment 17: The method of any one of claims 1-16, wherein the nucleic acid sequence encoding helper proteins sufficient for rAAV replication comprises a nucleotide sequence encoding an adenovirus helper (Ad helper) protein, optionally the nucleic acid sequence encoding helper proteins sufficient for rAAV replication comprises a nucleotide sequence encoding adenoviral helper proteins E2A and E4.

Embodiment 18: The method of any one of claims 1-17, wherein the titer of rAAV is at least 9.3 x 10¹³ vector genomes/3.0 × 10⁹ viable cells transfected.

Embodiment 19 The method of any one of claims 1-18, wherein the suspension of human embryonic cell line is progressively cultured in increasing volumes prior to transfection.

Embodiment 20: The method of any one of claims 1-19, wherein the culturing volumes are progressively increased from about 50 ml volumes to about 2000 liters volume.

Embodiment 21: The method of any one of claims 1-20, wherein the culturing volume comprises a concentration of an amino acid from about 1 mM to about 20 mM.

Embodiment 22: The method of any one of claims 1-21, wherein the culture media having a volume of about 5 liters comprises a concentration of an amino acid of about 10 mM.

Embodiment 23: The method of claims 21 or 22 wherein the amino acid is L-glutamine or L-alanyl-L-glutamine (Glutamax™).

Embodiment 21: The method of any one of claims 1-24, wherein the culture media having a volume of about 50 liters comprises: at least, from about 1 mM to about 20 mM L-glutamine, at least from about 0.01% to about 1% a nonionic, surfactant polyol or detergent and at least, from about 0.001% to about 1% of an anti-foaming agent

Embodiment 24: The method of claim 24, wherein the nonionic, surfactant polyol comprises pluronic acid.

Embodiment 26: The method of any one of claims 1-25, wherein the cultured human embryonic cell line comprises a cell density of about 3.0 × 10⁶ to about 1 × 10⁸ viable cells/ml.

Embodiment 27: The method of any one of claims 1-26, wherein the cultured human embryonic cell line comprises a cell density of about 4.0 × 10⁶ to about 6 × 10⁶ viable cells/ml or optionally to about 2.5 × 10⁷ viable cells/ml.

Embodiment 28: The method of any one of claims 1-27, further comprising: (i) adding about 1 liter of media to the transfected cells; and (ii) adding a cationic polymer at a ratio of between about 1:1 of polymer to DNA to about 3:1 of the polymer to DNA over a time course of about 10 minutes to about 60 minutes.

Embodiment 29: The method of claim 28, wherein the cationic polymer is added at a ratio of 2.2:1 of the polymer to DNA over a time course of about 1 minute to about 10 minutes.

Embodiment 30: The method of any one of claims 14-29, wherein the cationic polymer comprises a fully hydrolyzed linear polyethylenimine (PEI).

Embodiment 31: The method of any one of claims 1-30, wherein temperature of the culture media comprising the human embryonic cell suspension is increased to 37° C. at about 12 to 36 hours prior to transfection.

The method of claim 27, wherein the culture media is subjected to an air sparge at a flow rate between about 0.1 LPM to about 1.0 LPM.

The method of claim 27, wherein the culture media is subjected to an air sparge at a flow rate of about 0.5 LPM.

Embodiment 34: A method of producing a population of high titer recombinant adeno-associated virus (rAAV) lacking prokaryotic sequences comprising: (i) transfecting a mammalian cell line with a) a nucleic acid sequence encoding helper proteins sufficient for rAAV replication; b) a nucleic acid sequence encoding rep and cap genes, and c) a close ended linear duplexed rAAV vector nucleic acid comprising at least one ITR and a heterologous transgene operably linked to one or more regulatory elements, wherein the total amount of nucleic acid transfected from a), b), and c) per 1 × 10⁶ cells is less than 2 µg, e.g., less than 1 µg; (ii) culturing the transfected cells for at least 24 hours, e.g., for at least 40 hours; and (iii) harvesting the transfected cells and purifying the rAAV vector particles produced, wherein the titer of rAAV is at least 9.3 × 10¹³ vector genomes/3.0 × 10⁹ viable cells transfected.

Embodiment 35: The method of claim 34, wherein the mammalian cell line is a suspension cell line and the cells are transfected in suspension.

Embodiment 36: The method of any one of claims 34 or 35, wherein the cell line is derived from a human embryonic kidney cell line.

Embodiment 37: The method of any one of claims 34-36, wherein the mammalian cell line is a suspension adapted serum free cell line.

Embodiment 38: The method of any one of claims 34-37, wherein the ratio of a):b):c) is about 0.5-1.75: about 0.75-2.25: about 0.5-1.75 (weight:weight:weight), e.g., the ratio of a):b):c) is about 1:about 1-1.6: about 1 (weight:weight:weight).

Embodiment 39: The method of any one of claims 34-38, wherein a), b) and c) are transfected using a transfection composition comprising a), b) and c), and a stable cationic polymer, wherein the ratio of stable cationic polymer to total amount of nucleic acid from a), b) and c), is from about 1:1 to about 3:1 (weight/weight), e.g., the ratio of stable cationic polymer to total amount of nucleic acid from a), b) and c), is about 1.5:1.

Embodiment 40: The method of any one of claims 34-39, wherein each of a), b) and c) are provided on one or more close ended linear duplexed nucleic acid molecules.

Embodiment 41: The method of any one of claims 34-40, wherein the transfected nucleic acids a), b), and c) are synthetic nucleic acids and devoid of eukaryotic and prokaryotic cellular modifications of DNA.

Embodiment 42: The method of any one of claims 34-41, wherein a) the nucleic acid sequence encoding helper proteins sufficient for rAAV replication comprises a nucleotide sequence encoding an Ad helper protein, optionally the nucleic acid sequence encoding helper proteins sufficient for rAAV replication comprises a nucleotide sequence encoding adenoviral helper proteins E2A and E4.

Embodiment 43: The method of any one of claims 34-42, wherein, the amount of total of DNA from a), b) and c) are optionally 0.6, 0.7, 0.75, 0.8, 0.9, 1, 1.2, 1.4, 1.6 or 1.8 µg.

Embodiment 44: The method of any one of claims 34-43, wherein the suspension of the mammalian cell line is progressively cultured in increasing volumes of culture media prior to transfection.

Embodiment 45: The method of any one of claims 34-44, wherein the culturing volumes are progressively increased from about 50 ml volumes to about 2000 liter volumes.

Embodiment 46: The method of any one of claims 34-45, wherein the culturing volume comprises a concentration of an amino acid from about 1 mM to about 20 mM.

Embodiment 47: The method of any one of claims 34-46, wherein the culture media having a volume of about 5 liters comprises a concentration of an amino acid of about 10 mM.

Embodiment 48: The method of any one of claims 34-47, wherein the amino acid is L-glutamine.

Embodiment 49: The method of any one of claims 34-48, wherein the cells are in a culture volume of 50 to 100 liters.

Embodiment 50: The method of any one of claims 34-49, wherein the infectious particle titer is at least 3 × 10⁹ TCID50/ml.

Embodiment 51: The method of any one of claims 34-50, wherein the AAV Rep and the AAV Cap genes are from the same AAV serotype.

Embodiment 51: The method of any one of claims 34-50, wherein the AAV Rep and the AAV Cap genes are from different AAV serotypes.

Embodiment 53: The method of any one of claims 34-52, wherein the AAV ITR and the AAV Cap genes are from the same AAV serotype.

Embodiment 54: The method of any one of claims 34-52, wherein the AAV ITR and the AAV Cap genes are from different AAV serotypes.

Embodiment 55: The method of any one of claims 34-54, wherein the AAV ITR sequence is from AAV2 or from serotypes selected from the group consisting of AAV 1, 3a, 3b, 4, 5, 6, 7, 8, 9, 10, 11, and 13.

Embodiment 56: The method of any one of claims 34-55, wherein the AAV ITR sequence is synthetic.

Embodiment 57: A method of producing a population of purified recombinant adeno-associated virus (rAAV) that lacks prokaryotic sequences, comprising: (i) transfecting a mammalian cell line in suspended in culture media with a transfection composition; comprising: a) a nucleic acid sequence encoding helper proteins sufficient for rAAV replication, b) a nucleic acid sequence encoding rep and cap genes, c) a close ended linear duplexed rAAV vector nucleic acid comprising at least one ITR and a heterologous transgene operably linked to one or more regulatory elements, and d) a stable cationic polymer, and wherein the ratio of the stable cationic polymer to the total amount of nucleic acid contents from a), b) and c) is at least 1:1, e.g., the ratio of the stable cationic polymer to the total amount of nucleic acid contents from a), b) and c) is at least 1.5:1; (ii) culturing the transfected cell line for at least 24 hours, e.g., at least 40 hours; (iii) harvesting the transfected cell line of step (ii); and (iii) purifying the rAAV, wherein the purified virus has a particle to infectivity ratio is less than 2 × 10⁴ vg/TCID50.

Embodiment 57: The method of claim 57, wherein the mammalian cell line is a suspension cell line and the cells are transfected in suspension.

Embodiment 59: The method of any one of claims 57-58, wherein the mammalian cell line is derived from a human embryonic kidney cell line.

Embodiment 60: The method of any one of claims 57-59, wherein the human embryonic cell line is suspension-adapted, serum-free cell line derived from a human embryonic kidney cell line.

Embodiment 61: The method of any one of claims 57-60, wherein the ratio of the stable cationic polymer to the total amount of nucleic acid contents from a), b) and c) is from about 1.75:1 to about 2.75:1, e.g., the ratio of the stable cationic polymer to the total amount of nucleic acid contents from a), b) and c) is about 2:1.

Embodiment 62: The method of any one of claims 57-61, wherein the stable cationic polymer comprises a fully hydrolyzed linear polyethylenimine (PEI).

Embodiment 63: The method of any one of claims 57-62, wherein the stable cationic polymer comprises a fully hydrolyzed linear polyethylenimine and wherein the ratio of PEI to nucleic acid is selected from the group consisting of 1.2:1, 1.4:1, 1.6:1, 1.8:1, 2:1, 2.5:1, 2.8:1, and 2.2:1.

Embodiment 64: The method of any one of claims 57-63, wherein temperature of the culture media comprising the cell suspension is increased to 37° C. at about 12 to 36 hours prior to transfection.

Embodiment 65: The method of any one of claims 57-64, wherein the culture media is subjected to an air sparge at a flow rate between about 0.1 LPM to about 1.0 LPM.

Embodiment 66: The method of any one of claims 57-65, wherein the culture media is subjected to an air sparge at a flow rate of about 0.5 LPM.

Embodiment 66: The method of any one of claims 57-66, wherein the transfection composition is added to the suspended cells over a time course of about 10 minutes to about 60 minutes.

Embodiment 68: The method of any one of claims 57-67, wherein the culture media is added after step i) and before step ii).

Embodiment 69: The method of any one of claims 57-68, wherein the total amount of nucleic acid (DNA) from a), b) and c) is from about 1 µg to about 20 µg.

Embodiment 70: The method of any one of claims 57-69, wherein the total amount of DNA from a), b) and c) is from about 1 µg to about 10 µg.

Embodiment 71: The method of any one of claims 57-70, wherein the ratio of a):b):c) is about 0.5-1.75: about 0.75-2.25: about 0.5-1.75 (weight:weight:weight).

Embodiment 72: The method of any one of claims 57-71, wherein each of a), b) and c) are provided on one or more close ended linear duplexed nucleic acid molecules.

Embodiment 73: The method of any one of claims 57-72, wherein the transfected nucleic acids a), b), and c) are synthetic and devoid of eukaryotic and prokaryotic cellular modifications of DNA.

Embodiment 74: The method of any one of claims 57-73, wherein the ratio of a):b):c) is about 1:about 1-1.6: about 1 (weight:weight:weight).

Embodiment 75: The method of any one of claims 57-74, wherein a) the nucleic acid sequence encoding helper proteins sufficient for rAAV replication comprises a nucleotide encoding an Ad helper protein, optionally, the nucleic acid sequence encoding helper proteins sufficient for rAAV replication comprises a nucleotide sequence encoding adenoviral helper proteins E2A and E4.

Embodiment 76: The method of any one of claims 57-75, wherein the amount of total of DNA from a), b) and c) is 0.6, 0.7, 0.8, 0.9, 1, 1.2, 1.4, 1.6 or 1.8 µg.

Embodiment 77: The method of any one of claims 57-76, wherein the amount of total of DNA from a), b) and c); is about 0.75 µg.

Embodiment 78: The method of any one of claims 57-77, wherein the suspension of the mammalian cell line is progressively cultured in increasing volumes prior to transfection.

Embodiment 79: The method of any one of claims 57-78, wherein the culturing volumes are progressively increased from about 50 ml volumes to about 2000 liter volumes.

Embodiment 80: The method of any one of claims 57-79, wherein the culturing volumes are progressively increased from about 50 ml volumes to about 100 liter volumes.

Embodiment 81: The method of any one of claims 57-80, wherein the culturing volume comprises a concentration of an amino acid from about 1 mM to about 20 mM.

Embodiment 82: The method of any one of claims 57-81, wherein the culture media having a volume of about 5 liters comprises a concentration of an amino acid of about 10 mM.

Embodiment 83: The method of claim 81 or 82, wherein the amino acid is L-glutamine or L-alanyl-L-glutamine (Glutamax™).

Embodiment 84: The method of any one of claims 57-83, wherein the cells are in a culture volume of 50 litres to 100 liters.

Embodiment 85: The method of any one of claims 57-84, wherein the culture media having a volume of about 50 liters comprises: at least, from about 1 mM to about 20 mM L-glutamine, at least from about 0.01% to about 1% a nonionic, surfactant polyol or detergent and at least, from about 0.001% to about 1% of an anti-foaming agent

Embodiment 86: The method of claim 85, wherein the nonionic, surfactant polyol comprises pluronic acid.

Embodiment 87: The method of any one of claims 57-86, wherein the transfection composition comprises at least about 5% volume/volume (v/v) to about 20% v/v of the culture media.

Embodiment 88: The method of any one of claims 57-87, wherein the transfection composition comprises about 1 liter to about 5 liters of media.

Embodiment 89: The method of any one of claims 57-88, wherein the transfection composition comprises 5-50 % (volume/volume) of culture media.

Embodiment 90: The method of any one of claims 57-89, wherein the nucleic acid sequences added to the transfection comprise: about 0.1 µg to about 1 µg of Ad helper DNA, Rep/Cap DNA, or transgene per 0.5 × 10⁶ to about 5 × 10⁶ cells.

Embodiment 91: The method of any one of claims 57-90, wherein the AAV Rep and the AAV Cap genes are from same AAV serotype.

Embodiment 92: The method of any one of claims 57-90, wherein, the AAV Rep and the AAV Cap genes are from different AAV serotypes.

Embodiment 93: The method of any one of claims 57-92, wherein the AAV ITR and the AAV Cap genes are from same AAV serotype.

Embodiment 94: The method of any one of claims 57-92, wherein the AAV ITR and the AAV Cap genes are from different AAV serotypes.

Embodiment 95: The method of any one of claims 57-94, wherein the AAV ITR sequence is from AAV2 or from serotypes selected from the group consisting of AAV 1, 3a, 3b, 4, 5, 6, 7, 8, 9, 10, 11, and 13.

Embodiment 96: The method of any one of claims 57-95, wherein the Cap gene is from AAV8 serotype.

Embodiment 97: The method of any one of claims 1-96, wherein the packaged nucleic acid of the rAAV further lacks eukaryotic DNA sequences.

Embodiment 98: The method of any one of claims 1-97, wherein the closed ended linear duplexed nucleic acid comprises ½ of a protelomerase binding site.

Embodiment 99: The method of any one of claims 1-98, wherein the closed ended linear duplexed nucleic acid comprises ½ of a protelomerase binding site and wherein the ½ of a protelomerase binding site is formed by protelomerase digestion of a target binding site comprising a double stranded palindromic sequence of at least 10 base pairs in length.

Embodiment 100: A population of rAAV virions that lack prokaryotic DNA produced by the method of any one of claims 1-99.

Embodiment 101: A recombinant adeno-associated virus (rAAV) comprising a protelomerase target sequence.

Embodiment 102: The rAAV of claim 101, wherein the protelomerase target sequence comprises a double stranded palindromic sequence of at least 10 base pairs in length.

Embodiment 103: The rAAV of claim 101 or 102, further comprising a transgene. Exemplary additional aspects of the invention can be described by the following numbered Embodiments 1-74:

Embodiment 1: A method of producing a high titer population of recombinant adeno-associated virus (rAAV) lacking prokaryotic sequences, the method comprising: (i) inoculating a cell culture medium, optionally comprising cells of a host cell line, with a) a nucleic acid sequence encoding helper proteins sufficient for rAAV replication; b) a nucleic acid sequence encoding AAV rep and AAV cap genes, c) a close ended linear duplexed rAAV vector nucleic acid comprising at least one inverted terminal repeat (ITR) sequence and a heterologous transgene operably linked to one or more regulatory elements, and d) optionally, a polycationic polymer, wherein a ratio of stable cationic polymer to total amount of nucleic acid from a), b) and c), is from about 1:1 to about 3:1 (weight/weight), and optionally, a total amount of nucleic acids from a), b), and c) per 1 × 10⁶ host cells is less than about 2 µg; (ii) incubating the inoculated cell culture medium for a sufficient period of time to produce rAAV; and (iii) purifying the rAAV, and optionally, a titer of rAAV produced is higher than a titer of rAAV produced by a cell culture medium inoculated with a corresponding amount of a plasmid DNA (pDNA) comprising the heterologous transgene.

Embodiment 2: A method of producing a high titer population of recombinant adeno-associated virus (rAAV) lacking prokaryotic sequences, the method comprising: (i) transfecting cells of a host cell line in a culture medium with a) a nucleic acid sequence encoding helper proteins sufficient for rAAV replication; b) a nucleic acid sequence encoding AAV rep and AAV cap genes, and c) a close ended linear duplexed rAAV vector nucleic acid comprising at least one inverted terminal repeat (ITR) sequence and a heterologous transgene operably linked to one or more regulatory elements, wherein: (i) a total amount of nucleic acids from a), b), and c) per 1 × 10⁶ host cells is less than about 2 µg; or (ii) the host cells are transfected using a transfection composition comprising a), b) and c), and a polycationic polymer, wherein a ratio of polycationic polymer to total amount of nucleic acid from a), b) and c), is from about 1:1 to about 3:1 (weight/weight); (ii) incubating the transfected host cell line for a sufficient period of time to produce rAAV; (iii) optionally lysing the transfected host cells; and (iv) purifying the rAAV, and optionally, a titer of rAAV produced is higher than a titer of rAAV produced using a host cell line transfected with a corresponding amount of a pDNA comprising the heterologous transgene.

Embodiment 3: The method of Embodiment 1 or 2, wherein a virus titer of rAAV is at least 9.3 x 10¹³ vector genomes/3.0 x 10⁹ viable cells transfected.

Embodiment 4: The method of any one of Embodiments 1-3, wherein a virus titer of rAAV is at least 3.5 x 10¹¹ vp/ml.

Embodiment 5: The method of any one of Embodiments 1-4, wherein the purified rAAV has particle to infectivity ratio of less than 2 x 10⁴ vg/TCID50.

Embodiment 6: The method of any one of Embodiments 1-5, wherein said incubating the transfected host cell line is for at least about 24 hours.

Embodiment 7: The method of any one of Embodiments 1-6, wherein said incubating the transfected host cell line is for between about 40 hours to about 400 hours.

Embodiment 8: The method of any one of Embodiments 1-7, wherein the host cell line is in a cell culture volume of at least about 50 liters

Embodiment 9: The method of any one of Embodiments 1-8, wherein the host cell line is in a cell culture volume of from about 50 liters to about 100 liters.

Embodiment 10: The method of any one of Embodiments 1-9, wherein the total amount of the nucleic acids from a), b), and c) per 1 × 10⁶ cells is less than about 1.5 µg.

Embodiment 11: The method of any one of Embodiments 1-10, wherein the total amount of the nucleic acids from a), b), and c) per 1 × 10⁶ cells is less than about 1 µg.

Embodiment 12: The method of any one of Embodiments 1-11, wherein the total amount of the nucleic acids from a), b), and c) per 1 × 10⁶ cells is less than about 0.75 µg.

Embodiment 13: The method of any one of Embodiments 1-12, wherein the total amount of the nucleic acids from a), b), and c) per 1 × 10⁶ cells is at least about 0.25 µg.

Embodiment 14: The method of any one of Embodiments 1-13, wherein the total amount of the nucleic acids from a), b), and c) per 1 × 10⁶ cells is at least about 0.5 µg.

Embodiment 15: The method of any one of Embodiments 1-14, wherein a ratio nucleic acids of a):b):c) is about 0.5-1.75: about 0.75-2.25: about 0.5-1.75 (weight:weight:weight).

Embodiment 16: The method of any one of Embodiments 1-15, wherein a ratio nucleic acids of a):b):c) is about 0.75-1.5: about 1-1.75: about 0.75-1.25 (weight:weight:weight).

Embodiment 17: The method of any one of Embodiments 1-16, wherein a ratio nucleic acids of a):b):c) is about 1.4: about 1.5: about 1 (weight:weight:weight).

Embodiment 18: The method of any one of Embodiments 1-17, wherein the polycationic polymer is polyethylenimine (PEI).

Embodiment 19: The method of any one of Embodiments 1-18, wherein the polycationic polymer is linear polyethylenimine.

Embodiment 20: The method of any one of Embodiments 1-19 wherein the stable cationic polymer is fully hydrolyzed polyethylenimine.

Embodiment 21: The method of any one of Embodiments 1-20, wherein the ratio of the stable cationic polymer to total amount of nucleic acid from a), b) and c), is from about 1.5:1 to about 2.75:1.

Embodiment 22: The method of any one of Embodiments 1-21, wherein the ratio of the stable cationic polymer to total amount of nucleic acid from a), b) and c), is from about 1.9:1 to about 2.6:1.

Embodiment 23: The method of Embodiment any one of Embodiments 1-22, wherein the total amount of the nucleic acids from a), b), and c) per 1 × 10⁶ cells is from about 0.55 µg to about 0.75 µg and the ratio of the the ratio of the stable cationic polymer to total amount of nucleic acid from a), b) and c), is from about 2:1 to about 2.5:1.

Embodiment 24: The method of any one of Embodiments 1-23, wherein the host cell line is infected with a transfection composition volume of from about 5% to about 20% (volume/volume) of the host cell line culture volume.

Embodiment 25: The method of any one of Embodiments 1-24, wherein the host cell line is infected with a transfection composition volume of from about 7.5% to about 15% (volume/volume) of the host cell line culture volume.

Embodiment 26: The method of any one of Embodiments 1-25, wherein the transfection composition is added to the host cells over a time course of about 10 minutes to about 60 minutes.

Embodiment 27: The method of any one of Embodiments 1-26, wherein the method further comprises a step of culturing the host cell line for a period of time prior to transfecting the host cell line.

Embodiment 28: The method of Embodiment 27, wherein said culturing the host cell line comprises increasing the culturing volume from about 50 ml to about 2000 liters.

Embodiment 29: The method of Embodiment 27 or 28, wherein said culturing the host cell line comprises increasing the culturing volume from about 50 ml to about 100 liters.

Embodiment 30: The method of any one of Embodiments 1-29, wherein temperature of the culture medium comprising the host cell line is increased to 37° C. at about 12 hours to 36 hours prior to transfection.

Embodiment 31: The method of any one of Embodiments 1-30, wherein the culture medium comprises an amino acid at a concentration of from about 1 mM to about 20 mM.

Embodiment 32: The method of any one of Embodiments 1-31, wherein the culture medium comprises an amino acid at a concentration of about 5 mM to about 15 mM.

Embodiment 33: The method of any one of Embodiments 1-32, wherein the culture medium comprises an amino acid at a concentration of about 7.5 mM to about 12.5 mM

Embodiment 34: The method of any one of Embodiments 31-33, wherein the amino acid is L-glutamine or a dipeptide comprising L-glutamine.

Embodiment 35: The method of Embodiment 34, wherein the dipeptide comprising the L-glutamine is L-alanyl-L-glutamine.

Embodiment 36: The method of any one of Embodiments 1-35, wherein the culture medium comprises a nonionic, surfactant polyol or detergent at a concentration of from about 0.01% to about 1% (weight/volume).

Embodiment 37: The method of Embodiment 36, wherein the a nonionic, surfactant polyol comprises pluronic acid.

Embodiment 38: The method of any one of Embodiment 1-37, wherein the culture medium comprises an anti-foaming agent at a concentration of from about 0.001% to about 1% (weight/volume).

Embodiment 39: The method any one of Embodiments 1-38, wherein the culture medium is subjected to an air sparge at a flow rate between about 0.1 LPM to about 1.0 LPM.

Embodiment 40: The method any one of Embodiments 1-39, wherein the culture medium is subjected to an air sparge at a flow rate between about 0.25 LPM to about 0.75 LPM.

Embodiment 41: The method of any one of Embodiments 1-40, wherein the host cell line is a mammalian cell line.

Embodiment 42: The method of any one of Embodiments 1-41, wherein the host cell line is a human cell line.

Embodiment 43: The method of any one of Embodiments 1-42, wherein the host cell line is a human embryonic cell line.

Embodiment 44: The method of any one of Embodiments 1-43, wherein the host cell line is a human embryonic kidney cell line.

Embodiment 45: The method of any one of Embodiments 1-44, wherein the host cell line is a serum free cell line.

Embodiment 46: The method of any one of Embodiments 1-45, wherein the host cell line is suspension-adapted.

Embodiment 47: The method of any one of Embodiments 1-46, wherein the host cell line is suspended in the culture medium.

Embodiment 48: The method of any one of Embodiments 1-47, wherein cells of the host cell line are transfected in suspension.

Embodiment 49: The method of any one of Embodiments 1-48, wherein the host cell line comprises a cell density of about 3.0 × 10⁶ to about 1 × 10⁸ viable cells/ml.

Embodiment 50: The method of any one of Embodiments 1-49, wherein the host cell line comprises a cell density of about 4.0 × 10⁶ to about 6 × 10⁶ viable cells/ml.

Embodiment 51: The method of any one of Embodiments 1-50, wherein the host cell line comprises a cell density of about 2.5 × 10⁷ viable cells/ml.

Embodiment 52: The method of any one of Embodiments 1-51, wherein at least one of the nucleic acids a) and b) is comprised in a close ended linear duplexed nucleic acid molecule.

Embodiment 53: The method of any one of Embodiments 1-52, wherein each of the nucleic acids a) and b) independently are comprised in a close ended linear duplexed nucleic acid molecule.

Embodiment 54: The method of any one of Embodiments 1-53, wherein the closed ended linear duplexed nucleic acid comprises ½ of a protelomerase binding site.

Embodiment 55: The method of any one of Embodiments 1-54, wherein the closed ended linear duplexed nucleic acid comprises ½ of a protelomerase binding site, and wherein the ½ of the protelomerase binding site is formed by protelomerase digestion of a target binding site. Comprising a double-stranded palindromic sequence of at least 10 base pairs in length.

Embodiment 56: The method of any one of Embodiments 1-55, wherein the AAV rep and AAV cap genes are from same serotypes.

Embodiment 57: The method of any one of Embodiments 1-56, wherein the AAV rep and AAV cap genes are from different serotypes.

Embodiment 58: The method of any one of Embodiments 1-57, wherein the AAV ITR sequences and AAV cap gene are from same serotypes.

Embodiment 59: The method of any one of Embodiments 1-58, wherein the AAV ITR sequences and AAV cap gene are from different serotypes.

Embodiment 60: The method of any one of Embodiments 1-59, wherein the AAV ITR sequences and AAV rep gene are from same serotypes.

Embodiment 61: The method of any one of Embodiments 1-60, wherein the AAV ITR sequences and AAV rep gene are from different serotypes.

Embodiment 62: The method of any one Embodiments 1-61, wherein the AAV rep gene is from a serotype selected from the group consisting of AAV1, 2, 3a, 3b, 4, 5, 6, 7, 8, 9, 10, 11 and 13.

Embodiment 63: The method of any one of Embodiments 1-62, wherein the AAV rep gene is from a serotype selected from the group consisting of AAV2, 3a, 3b, 8, 9 and 10.

Embodiment 64: The method of any one Embodiments 1-63, wherein the AAV cap gene is from a serotype selected from the group consisting of AAV1, 2, 3a, 3b, 4, 5, 6, 7, 8, 9, 10, 11 and 13.

Embodiment 65: The method of any one of Embodiments 1-64, wherein the AAV cap gene is from a serotype selected from the group consisting of AAV2, 3a, 3b, 8, 9 and 10.

Embodiment 66: The method of any one of Embodiments 1-65, wherein the AAV ITR sequences are from serotypes independently selected from AAV1, 2, 3a, 3b, 4, 5, 6, 7, 8, 9, 10, 11 and 13.

Embodiment 67: The method of any one of Embodiments 1-66, wherein the AAV ITR sequences are from serotypes independently selected from AAV2, 3a, 3b, 8, 9 and 10.

Embodiment 68: The method of any one of Embodiments 1-67, wherein the AAV ITR sequence is a synthetic sequence.

Embodiment 69: The method of any one of Embodiments 1-68, wherein the nucleic acid sequence encoding helper proteins sufficient for rAAV replication comprises a nucleotide sequence encoding an adenoviral helper protein.

Embodiment 70: The method of any one of Embodiments 1-69, wherein the nucleic acid sequence encoding helper proteins sufficient for rAAV replication comprises a nucleotide sequence encoding adenoviral helper proteins E2A and E4.

Embodiment 71: The method of any one of Embodiments 1-70, wherein at least one of the transfected nucleic acid is a synthetic nucleic acid and devoid of eurkaryotic and prokaryotic cellular modifications of DNA.

Embodiment 72: The method of any one of Embodiments, 1-71, wherein the rAAV further lacks eukaryotic DNA sequences.

Embodiment 73: The method of any one of Embodiments 1-72, wherein said incubating for a sufficient period of time to produce rAAV is for a period of at least 24 hours.

Embodiment 74: A population of rAAV virions produced by the method of any one of Embodiments 1-73.

It is to be understood that one, some, or all of the properties of the various embodiments described herein may be combined to form other embodiments of the present invention. While various embodiments of the present invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the present invention should not be limited by any of the above described embodiments.

### Definitions

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ± 100% in some embodiments ± 50%, in some embodiments ± 20%, in some embodiments ± 10%, in some embodiments ± 5%, in some embodiments ±1%, in some embodiments ± 0.5%, and in some embodiments ± 0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

As used herein, the singular forms “a”, “an” and “the” are intended to include the plural forms as well, unless the context clearly indicates otherwise. Furthermore, to the extent that the terms "including", "includes", "having", "has", "with", or variants thereof are used in either the detailed description and/or the claims, such terms are intended to be inclusive in a manner similar to the term "comprising." It must be noted that as used herein and in the appended claims, the singular forms "a," "an," and "the" include the plural reference unless the context clearly dictates otherwise. Thus, for example, a reference to a “protein” is a reference to one or more proteins, and includes equivalents thereof known to those skilled in the art and so forth.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements--or, as appropriate, equivalents thereof--and that other elements can be included and still fall within the scope/definition of the defined item, composition, apparatus, method, process, system, etc.

As used herein, the term “helper virus” or “contaminating helper virus” refers to a virus used when producing copies of a helper virus-dependent viral vector, such as adeno-associated virus, which does not have the ability to replicate on its own. The helper virus is used to co-infect cells alongside the viral vector and provides the necessary proteins for replication of the genome of the viral vector. The term encompasses intact viral particles, empty capsids, viral DNA and the like. Helper viruses commonly used to produce rAAV particles include adenovirus, herpes simplex virus, cytomegalovirus, Epstein-Barr virus, and vaccinia virus.

Helper viruses include Adenovirus (AV), and herpes simplex virus (HSV), and systems exist for producing AAV in insect cells using baculovirus. It has also been proposed that papilloma viruses may also provide a helper function for AAV (See, e.g., Hermonat et al., Molecular Therapy 9, S289-S290(2004)). Helper viruses include any virus capable of creating an allowing AAV replication. AV is a nonenveloped nuclear DNA virus with a double-stranded DNA genome of approximately 36 kb. AV is capable of rescuing latent AAV provirus in a cell, by providing E1a, E1b55K, E2a, E4orf6, and VA genes, allowing AAV replication and encapsidation. HSV is a family of viruses that have a relatively large double-stranded linear DNA genome encapsidated in an icosahedral capsid, which is wrapped in a lipid bilayer envelope. HSV are infectious and highly transmissible. The following HSV-1 replication proteins were identified as necessary for AAV replication: the helicase/primase complex (UL5, UL8, and UL52) and the DNA binding protein ICP8 encoded by the UL29 gene, with other proteins enhancing the helper function.

The term “non-adherent cell line” or “suspension cell line”, as used herein, refers to a cell line that is able to survive in a suspension culture without being attached to a surface (e.g. tissue culture plastic carrier or micro-carrier). The adaptation to a non-adherent cell line is a prolonged process requiring passaging with diminishing amounts of serum, thereby selecting an irreversibly modified cell population. The cell line can be grown to a higher density than adherent conditions would allow and is, thus, more suited for culturing in an industrial scale, e.g. in a bioreactor setting or in an agitated culture.

As used in this specification and the appended claims, the term “or” is generally employed in its sense including “and/or” unless the content clearly dictates otherwise.

The term “promoter” as used herein is defined as a DNA sequence recognized by the synthetic machinery of the cell, or introduced synthetic machinery, required to initiate the specific transcription of a polynucleotide sequence. A “constitutive” promoter is a nucleotide sequence which, when operably linked with a polynucleotide which encodes or specifies a gene product, causes the gene product to be produced in a cell under most or all physiological conditions of the cell. An “inducible” promoter is a nucleotide sequence which, when operably linked with a polynucleotide which encodes or specifies a gene product, causes the gene product to be produced in a cell substantially only when an inducer which corresponds to the promoter is present in the cell. A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

A “protelomerase” target sequence is any DNA sequence whose presence in a DNA template allows for its conversion into a closed linear DNA by the enzymatic activity of protelomerase. In other words, the protelomerase target sequence is required for the cleavage and religation of double stranded DNA by protelomerase to form covalently closed linear DNA. Typically, a protelomerase target sequence comprises any perfect palindromic sequence i.e any double-stranded DNA sequence having two-fold rotational symmetry, also described herein as a perfect inverted repeat. The length of the perfect inverted repeat differs depending on the specific organism. In Borrelia burgdorferi, the perfect inverted repeat is 14 base pairs in length. In various mesophilic bacteriophages, the perfect inverted repeat is 22 base pairs or greater in length. Also, in some cases, e.g. E. coli N15, the central perfect inverted palindrome is flanked by inverted repeat sequences, i.e. forming part of a larger imperfect inverted palindrome.

As used herein, the terms “recombinant AAV (rAAV) vector” or “gene delivery vector” refer to a virus particle that functions as a nucleic acid delivery vehicle, and which comprises the vector genome (e.g., viral DNA [vDNA]) packaged within an AAV capsid. Alternatively, in some contexts, the term “vector” may be used to refer to the vector genome/vDNA alone.

A “rAAV vector genome” or “rAAV genome” is an AAV genome (i.e., vDNA) that comprises one or more heterologous nucleotide sequences. rAAV vectors generally require only the 145 base terminal repeat(s) (TR(s)) in cis to generate virus. All other viral sequences are dispensable and may be supplied in trans (Muzyczka, (1992) Curr. Topics Microbiol. Immunol. 158:97). Typically, the rAAV vector genome will only retain the minimal TR sequence(s) so as to maximize the size of the transgene that can be efficiently packaged by the vector. The structural and non- structural protein coding sequences may be provided in trans (e.g., from a vector, such as a plasmid, or by stably integrating the sequences into a packaging cell). The rAAV vector genome comprises at least one TR sequence (e.g., AAV TR sequence, synthetic, or other parvovirus TR sequence), optionally two TRs (e.g., two AAV TRs), which typically will be at the 5' and 3' ends of the heterologous nucleotide sequence(s), but need not be contiguous thereto. The TRs can be the same or different from each other.

The term “terminal repeat” or “TR” includes any viral terminal repeat and synthetic sequences that form hairpin structures and function as an inverted terminal repeat (ITR), such as the “double-D sequence” as described in U.S. Pat. No. 5,478,745 to Samulski et al. The capsid structures of autonomous parvoviruses and AAV are described in more detail in BERNARD N. FIELDS et al., VIROLOGY, volume 2, chapters 69 & 70 (4th ed., Lippincott-Raven Publishers). See also, description of the crystal structure of AAV2 (Xie et al., (2002) Proc. Nat. Acad. Sci. 99: 10405-10), AAV4 (Padron et al., (2005) I. Virol. 79: 5047-58), AAV5 (Walters et al., (2004) I. Virol. 78: 3361-71) and CPV (Xie et al., (1996) I. Mol. Biol. 6:497-520 and Tsao et al., (1991) Science 251 : 1456-64).

An “AAV terminal repeat” or “AAV TR” may be from any AAV, including but not limited to serotypes AAV-1, AAV-2, AAV-3, AAV-4, AAV-5, AAV-6, AAV-7, AAV-8, AAV-9, AAV-10, AAV-11, AAV-12, AAV-13, or any other AAV now known or later discovered. The AAV terminal repeats need not have a wild-type terminal repeat sequence (e.g., a wild-type sequence may be altered by insertion, deletion, truncation or missense mutations), as long as at least one of the terminal repeat mediates the desired functions, a functional TR, e.g., replication, virus packaging, integration, and/or provirus rescue, and the like. One of skill in the art understands to choose a Rep protein that is functional for replication of the functional TR.

A “transgene” is used herein to conveniently refer to a polynucleotide or a nucleic acid that is intended or has been introduced into a cell or organism. Transgenes include any nucleic acid, such as a gene that encodes a polypeptide or protein. Suitable transgenes, for example, for use in gene therapy are well known to those of skill in the art. For example, the vectors described herein can deliver transgenes and uses that include, but are not limited to, those described in U.S. Pat. Nos. 6,547,099; 6,506,559; and 4,766,072; Published U.S. Application No. 20020006664; 20030153519; 20030139363; and published PCT applications of WO 01/68836 and WO 03/010180, and e.g. miRNAs and other transgenes of WO2017/152149; each of which are hereby incorporated herein by reference in their entirety.

The term “tropism” as used herein refers to preferential entry of the virus into certain cells or tissues, optionally followed by expression (e.g., transcription and, optionally, translation) of a sequence(s) carried by the viral genome in the cell, e.g., for a recombinant virus, expression of a heterologous nucleic acid(s) of interest.

The term “variant,” when used in the context of a polynucleotide sequence, may encompass a polynucleotide sequence related to a wild type gene. This definition may also include, for example, “allelic,” “splice,” “species,” or “polymorphic” variants. A splice variant may have significant identity to a reference molecule, but will generally have a greater or lesser number of polynucleotides due to alternate splicing of exons during mRNA processing. The corresponding polypeptide may possess additional functional domains or an absence of domains. Species variants are polynucleotide sequences that vary from one species to another. Of particular utility in the invention are variants of wild type gene products. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.1, 2.2, 2.7, 3, 4, 5, 5.5, 5.75, 5.8, 5.85, 5.9, 5.95, 5.99, and 6. This applies regardless of the breadth of the range.

All documents mentioned herein are incorporated herein by reference. The contents of all references, patents, and published patent applications cited throughout this application, as well as the figures and the sequence listing, are hereby incorporated by reference for all purposes to the same extent as if each individual publication or patent document were so individually denoted. By their citation of various references in this document, applicants do not admit any particular reference is “prior art” to their invention. Embodiments of inventive compositions and methods are illustrated in the following examples.

### EXAMPLES

The following non-limiting Examples serve to illustrate selected embodiments of the invention which do not limit the scope of the invention described in the claims. It will be appreciated that variations in proportions and alternatives in elements of the components shown will be apparent to those skilled in the art and are within the scope of embodiments of the present invention.

### Example 1: AAV Production Using Closed Linear (cl) DNA

The purpose of this study was to assess large scale production of rAAV.

### Materials and Methods

TCID50 assay: The infectious titer (TCID50) method is used to evaluate the in vitro AAV infectivity of drug product in HeLa RC32 cells. In this assay, HeLa RC32 cells are transduced with adenovirus type 5 helper virus and serial dilutions of drug product. After three days of infection the cells are treated with proteinase K to digest protein and the replicated AAV vector DNA is quantitated with qPCR technology. This method utilizes a DNA primer and fluorescent dye-based detection system. The absolute quantity of the ITR target sequence from the vector DNA is interpolated from a standard curve prepared with a plasmid. Containing ITR is prepared as a test sample and is used as an assay control. Results are expressed as infectious units per milliliter (IU/mL). It is noted that for comparing TCID50/ml among different preparations, TCID50/ml is preferably normalized to vg/ml.

Table 1

Description of analytical testing and specifications to be completed for the 50 L scale vector productions

| | |
|---|---|
| Assay | Specification |
| Western Blot | Presence of bands corresponding to VP1, VP2 and VP3 capsid proteins compared to molecular weight standard and control |
| Silver Stain | VP1, VP2 and VP3 capsid proteins are the dominant protein bands compared to molecular weight standard and control |
| Vector Genome Sequencing | Sequence matches 100% |
| Genome integrity (alkaline gel) | ≥75% full-length (densitometry) Co-migrates with Transgene clDNA or matches expected restriction size of the vector DNA from the plasmid pre-cursor. Size is confirmed versus a DNA marker. |
| Residual plasmid backbone (Multiplex qPCR) | None detected, results are reported as pg DNA/1 × 10⁹ vg |
| UV 260/280 | 1.20 to 1.40 |
| Replication Competent AAV | <1 rcAAV / 3.0 × 10¹⁰ vg |

The PRO10™ cell line (AskBio, NC, USA) used to manufacture recombinant adeno-associated viral vectors (rAAV) is a suspension-adapted, serum-free cell line derived from the human embryonic kidney cell line 293 (HEK293). The PRO10™ Viral vector manufacture is a batch process carried out at mid- to high-range cell densities and employs a triple transfection method via condensation of the requisite plasmid (pDNA) or closed linear (cl) DNA substrate with linear Polyethylenimine MAX in a cocktail of production media. Both cell growth and production medias are chemically defined with no animal derived components. Each DNA molecule provides a key element for the recombinant AAV production. The first provides Adenovirus helper (Ad helper) proteins for efficient replication and packaging of the vector but lacks essential Adenoviral structural and replication genes to generate an Adenovirus. The second is an AAV8 or, AAVrh10 Trans construct (packaging construct) containing the AAV2 rep gene and AAV8 or, AAVrh10 capsid (cap) protein gene. The third construct is the therapeutic transgene encoding, AAV vector construct and contains the adeno-associated virus 2 inverted terminal repeat (ITR) sequences flanking (5' to 3') the gene of interest. The construct used for all experiments was the dual GFP and Luciferase reporter. Additionally, subsequent studies utilized two therapeutic transgene cassettes comprising CYP and GAA transgenes.

Initial experiments were conducted applying Design of Experiments (DoE) methodology in a traditional, non-block approach at bench scale (31.25 mL - 2 L) to identify and optimize critical parameters relating to production by simultaneously examining the factors clDNA concentration, ratio of clDNA to transfection reagent. All small-scale experiments were controlled by side-by-side vector production using an optimized triple-plasmid transfection system. Additional factors that will be evaluated include, but are not limited to, media, cell density, time of transfection, transfection volume, temperature, and other cell-dependent or cell-independent factors.

Small-scale transfected cultures were incubated for approximately 72 hrs post-transfection (hpt) and then harvested by mechanical cell lysis. Total vector production was assessed via vector genome (vg) quantification using the in-house qPCR-based DNase Resistant Particle (DRP) method specific to the viral ITRs. Yields typically range from 4-6 × 10¹¹ vg/mL, as indicated by qPCR Yields were further assessed by observing transgene-targeted qPCR as well as total viral particle (capsids) per mL (vp/mL) via ELISA. Relative packaging efficiency is also modeled by observing the A260/280 ratio at harvest of affinity-purified lysates via SEC-HPLC.

The primary aim of the small-scale screening experiments was to identify near-optimal transfection conditions for the 50 L scaled portion of the experimental plan. For both the pDNA and clDNA runs, cells were thawed, cultured and progressively expanded until inoculation into the 50 L production bioreactor. The cell culture expansion process continued in the production bioreactor prior to transient transfection being performed. The transfected cell culture was incubated in the production bioreactor for approximately 72-hpt. At harvest, the transfected cell culture was lysed and clarified via depth and membrane filtration followed by purification. Purification consists of capture chromatography, gradient ultracentrifugation, ion exchange chromatography, ultrafiltration/diafiltration (UF/DF), and a 0.2 µm filtration step. Table 3 provides characterization testing for rAAV vector produced by pDNA and clDNA, respectively.

### Detailed Process Description for 50L SUB Upstream Operations

To generate a 50 L batch, cells were thawed, cultured and progressively expanded until inoculation into the 50 L production bioreactor. The cell culture expansion process continued in the production bioreactor prior to transient transfection being performed. Currently, the seed train growth media is supplemented with L-Glutamine to a final concentration of 10 mM, which is used for recovery of frozen cell stocks as well as inoculum expansion up to 5 L suspensions using a 10 L WAVE bag bioreactor. The media used in the WAVE suspension was supplemented with 0.2% PLURONIC™ acid. The growth media used following seed of the ThermoFisher 50 L single-use, stirred-tank bioreactor (SUB, STR) is composed of the see train growth media supplemented with about 1 to 100 mM GLUTAMAX™, about 0.01% to 10% PLURONIC™ acid (ThermoFisher, Waltham, MA), and about 0.001% to 1% FOAMAWAY™ (Gibco, Waltham, MA). GLUTAMAX™ is a stabilized dipeptide source of L-glutamine designed to prevent degradation and reduce toxic buildup of excess ammonia.

Transient transfection to produce AAV was carried out at cell densities between 3.25 - 4.25 × 10⁶ viable cells/mL³ via condensation of three clDNA and linear Polyethylenimine MAX (Polysciences Inc., Warrington, PA) (PEI Max). The transfection cocktail constitutes 10% (v/v) of the culture volume (5 L). Condensation was carried out in a custom 10 L WAVE Rocker bag equipped with tubing mated for the 50 L SUB. The transfection cocktail was prepared by first adding 4 L of media to the rocker bag at 25° C. with gentle rocking (8° angle, 25 RPM). To prevent the bag from deflating, an air overlay is applied at 0.2 LPM. The plasmids (Table 2) were then added, followed by a 1 L chase with media.

Table 2

Ratio of each clDNA used normalized to the cell density at the time of transfection.

| | |
|---|---|
| Construct | Ratio |
| Ad helper | 0.15 to 0.32 µg DNA/ 1 × 10⁶ cells |
| Rep/Cap | 0.15 to 0.32 µg DNA/ 1 × 10⁶ cells |
| Transgene | 0.15 to 0.32 µg DNA/ 1 × 10⁶ cells |

Following the media chase, PEI was added over the course of 1 minute and chased with 1L of media. The cocktail was incubated for 7 minutes, and then transferred to the SUB. The transfection-cell suspension is incubated for three hours and quenched by a 10% (v/v) volume of chemically defined, serum-free HEK293 media supplemented with 10 mM L-Glutamine.

### SUB Control Parameters

The current large-scale manufacturing platform utilized a Finesse G3Pro Universal Controller outfitted with a ThermoFisher jacketed 50 L SUB. The single-use vessels were equipped with a 3-blade, 45° pitch, axial impellor, dual-sparger (Frit-Drilled-Hole) design, along with primary Finesse TruFluor pH/DO single-use probe sheaths as well as secondary Pall Kleenpak connections for reusable pH/DO probe inserts. The day before media charge, the bag was installed and inflated with an air overlay at 10 LPM. The optical/reusable DO probe was connected to the transmitter. On the day of charge, the DO probe was calibrated using a 2-pt slope calibration. Following media addition, both single-use and reusable pH probes were standardized using an offline sample on a calibrated blood-gas analyzer.

The SUB temperature was ramped to 37° C. the day before inoculation. The media was then conditioned by saturating with a continuous drilled-hole air sparge at a flow rate of 0.5 LPM (0.025 VVM). Prior to inoculation, both single-use and reusable DO probes were standardized to 100% air saturation using a 1 pt calibration.

Following inoculation, the controller was set to administer a continuous drilled-hole air sparge at a rate of 0.5 LPM, and the headspace was swept with an air overlay of 1 LPM. DO was controlled via O₂ gas cascade and designed to maintain the set point by increasing O₂ flow rate to the frit sparger from 0.00 to 5.00LPM (0-100% DO output / 0-100% MFC-3 output). pH was controlled on the high end (7.0 -14) by increasing CO₂ gas flow to the frit sparger from 0.00 to 2.00 LPM (0-(-100)%) output / 0-100% MFC-4 output); however, a base supply was not used to control pH on the low end, but rather, it was allowed to drift naturally.

### Results

### DoE Evaluation of Total clDNA (µg)Per 1 × 10⁶ Viable Cells and PEI_{:}DNA Ratio

In a DoE setting, µg DNA per 1 × 10⁶ viable cells and PEI:DNA ratio were studied in a range between 0.5 - 2 µg and 1 - 3, respectively. The design was a custom response surface model (RSM) with three levels for each factor allowing for the interpretation of both linear and quadratic effects. Duplicate center points in the design space were used to estimate the significance of each effect.

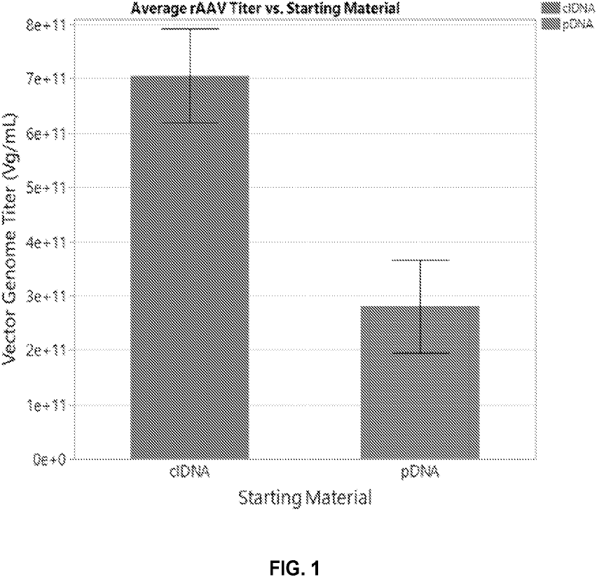

Total vector production at harvest was evaluated via ITR-qPCR (FIG. 1). The data indicates 2-2.5 times increase in specific (vg/cell) productivity using the clDNA as starting material compared to the pDNA as starting material.

Excluding the plasmid values, the fit model was used to model responses to identify significant and interacting factors as well as to discern the optimal clDNA conditions for transfection. The results of the JMP analysis are summarized below:

Table 3

Summary of Fit

| | |
|---|---|
| R Square | 0.941275 |
| RSquare Adj | 0.911913 |
| Root Mean Square Error | 4.87e+10 |
| Mean of Response | 2.59e+11 |
| Observations (or Sum Wgts) | 1 6 |

Table 4

Analysis of Variance

| Source | DF | Sum of Squares | Mean Square | F Ratio |
|---|---|---|---|---|
| Model | 5 | 3.8018 × 10²³ | 7.604 × 10²² | 32.0572 |
| Error | 10 | 2.3719x 10²² | 2.372 × 10²¹ | Prob>F |
| C. Total | 15 | 4.039 × 10²³ | | <0.0001* |

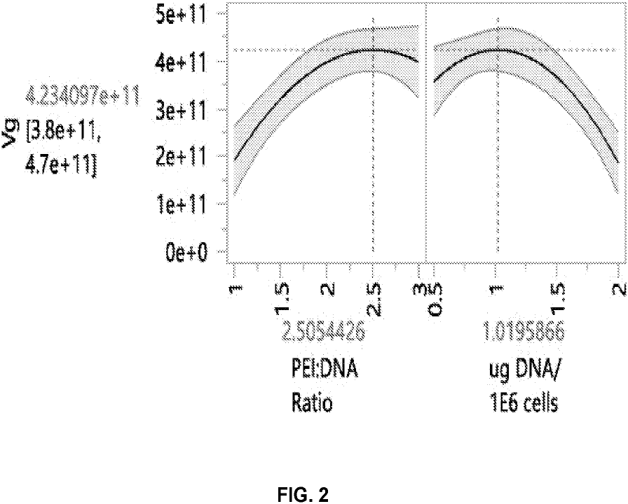

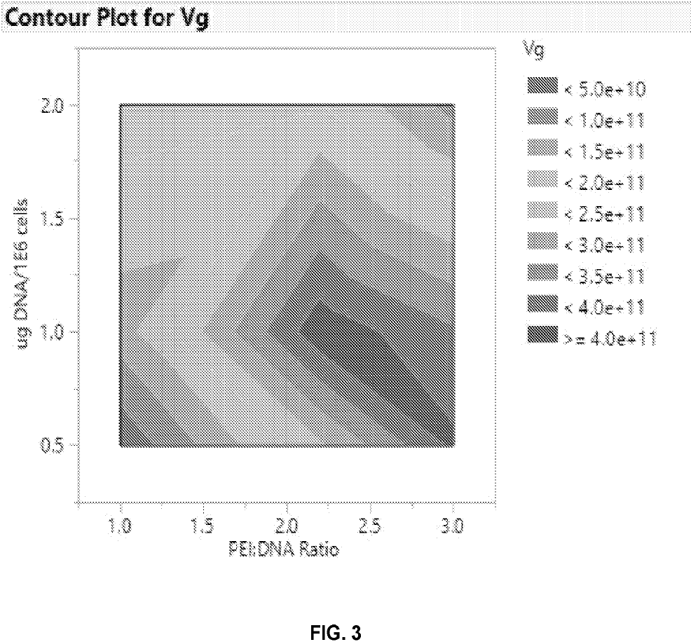

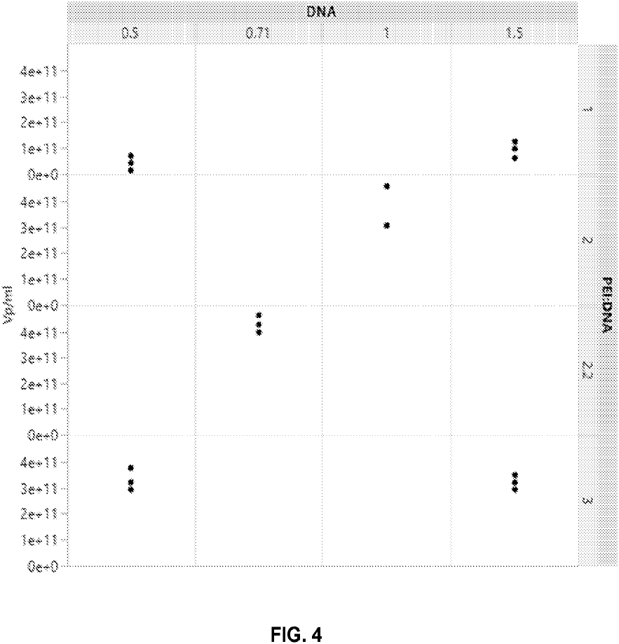

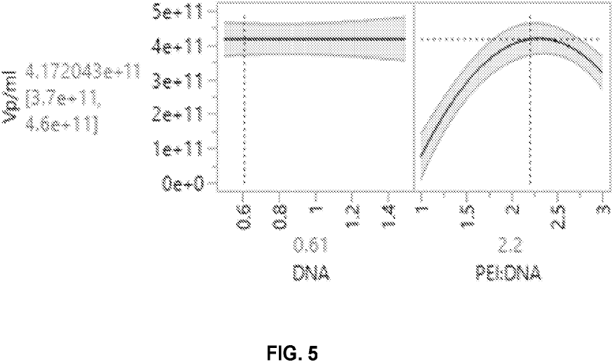

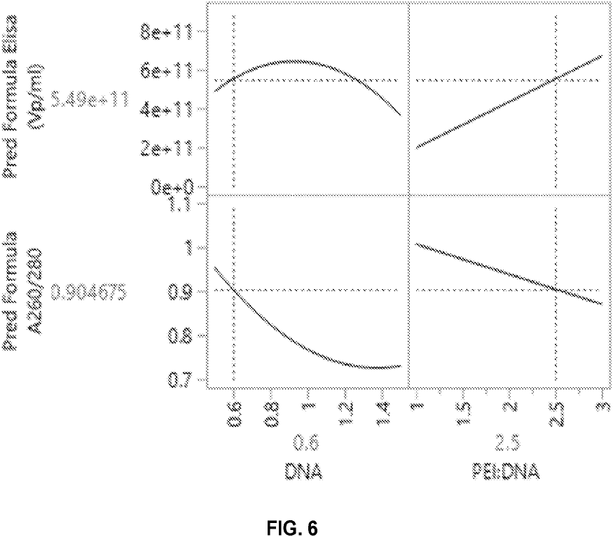

Summary of fit and analysis of variance for the factors µg clDNA per 1 × 10⁶ cells (total clDNA) and PEI:DNA ratio were analyzed (FIG. 2). The regression model accounts for much of the variation observed in the experiment. The R² was 0.941 indicating that less than 6% of the variation in vg titer cannot be explained by the factor of total clDNA or PEI:DNA A contour plot displayed the effects of clDNA and PEI:DNA ratio on vector genome titer in cell lysate, expressed in vg/mL(FIG. 3). As shown in FIG. 4, the clDNA used to generate recombinant AAVrh10CYP suggest less than lug DNA e.g between 0.6-0.7 ug required to achieve high titer of AAV. Furthermore, FIG. 5 and FIG. 6. Respectively show PEI:DNA as 2.2 and 2.5 to generate recombinant AAVrh10CYP and AAV8GAA and the optimal clDNA was 0.6 ug considering both overall yield and packaging efficiency.

Table 5

Parameter Estimates

| Term | Estimate | Std Error | t Ratio | Prob>\|t\| |
|---|---|---|---|---|
| Intercept | 4.049 × 10¹¹ | 2.38 × 10¹⁰ | 17.04 | <0.0001* |
| PEI:DNA Ratio(1,3) | 6.25 × 10¹⁰ | 1.65 × 10¹⁰ | 3.78 | 0.0036* |
| PEI:DNA Ratio*PEI:DNA Ratio | -1.02 × 10¹¹ | 2.99 × 10¹⁰ | -3.42 | 0.0066* |
| µg DNA/1 × 10⁶ cells(0.5, 2) | -1.77 × 10¹⁰ | 1.66 × 10¹⁰ | -1.07 | 0.3117 |
| µg DNA/1 × 10⁶ cells^{∗}µg | -1.38 × 10¹¹ | 3.28 × 10¹¹ | -4.21 | 0.0018* |
| DNA/1 × 10⁶ cells PEI:DNA Ratio^{∗}µg DNA/1 × 10⁶ cells | -1.33 × 10¹¹ | 1.80 × 10¹⁰ | -7.26 | <0.0001* |

### 50L SUB Runs

For each 50 L lot, QC assays were performed on in-process samples, purified bulk and final product and release testing of product. The following QC assays were performed and the results therein:

Table 6

Analytical testing, specification and results for pDNA- and clDNA-derived vector

| Assay | Analytical Method | Specification | Result pDNA | Result clDNA |
|---|---|---|---|---|
| Strength | | | | |
| Infectious particles titer | TCID50 | Report Result | 1.65 × 10¹⁰ TCID50/mL | 3.56 × 10⁹ TCID50/mL |
| Particle/Infectivit y Ratio (vg/TCID50) | - | Report Result | 9.7 × 10³ | 1.5 × 10⁴ |
| Genome integrity (alkaline gel) | Restriction analysis | ≥75% full-length (densitometry) Co-migrates with Transgene clDNA or matches expected restriction size of the vector DNA from the plasmid pre-cursor. Size is confirmed versus a DNA marker. | Conforms to expected vector genome size | Conforms to expected vector genome size |
| Quality | | | | |
| Osmolality (mOsm/Kg) | EP 2.2.35 | Report Result | 1022 mOsm/Kg water | 1007 mOsm/Kg water |
| pH | Potentiometry | Report Result | 7.08 | 7.05 |

Table 7

TCID50/ml and particle to infectivity (vg/TCID50) ratio results for pDNA- and clDNA-derived vector.

| | TCID50/ml | Particle to infectivity ratio (vg/TCID50) |
|---|---|---|
| pDNA control | 6.32E+08 | 1106.8 |
| DoE clDNA | 1.36E+09 | 521.8 |
| DoE clDNA | 6.32E+08 | 983.47 |
| 50 L pDNA | 1.65E+10 | 9700 |
| 50 L clDNA | 2.94E+10 | 2425.4 |
| 50 L clDNA | 1.36E+10 | 6605.1 |
| 50 L clDNA | 3.56E+09 | 15000 |

As shown in Table 7, in DoE experiments and in 50L runs, clDNA derived vectors show increased infectivity compared to pDNA controls as indicated by lower vg/TCID50 ratio for clDNA compared to that of pDNA

### Conclusion

It was demonstrated that the clDNA system can be used to produce AAV, however, consistent batch to batch small scale manufacturing runs along with linearly scaled vector production remains to be demonstrated with other serotypes and transgene constructs as has been done with plasmid DNA. The quadratic effect on total clDNA noted in the above experiments was also observed in similar experiments used to optimize the pDNA transfection process. Further experiments revealed potential of a product-specific relationship with respect to total clDNA and PEI, remains to be evaluated further. In order to address the small and larger scale production variations, additional studies will be carried out. These studies will be focused on establishing stability of the clDNA starting material, handling of clDNA prior to transfection, assessing PEI:clDNA ratio, clDNA ratio and PEI: clDNA complexation kinetics.

Aside from yield and strength, the analytical test results from the scaled 50L runs were consistent with one another. Assay results for purity, safety, quality, and identity were highly similar, regardless of starting material.

Future studies are planned to understand gaps in yield, packaging efficiency as well as purity and potency in scaled vector preps as well as further optimization work.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of producing a recombinant adeno-associated virus (rAAV) lacking prokaryotic sequences comprising:
culturing a human embryonic cell line in suspension;
transfecting the human embryonic cell line with a) a nucleic acid sequence encoding helper proteins sufficient for rAAV replication; b) a nucleic acid sequence encoding AAV rep and AAV cap genes, and c) a close ended linear duplexed rAAV vector nucleic acid comprising at least one inverted terminal repeat (ITR) sequence and a heterologous transgene operably linked to one or more regulatory elements;
incubating the transfected human cell line for between about 40 to 400 hours; and
optionally, lysing the transfected human cell line and purifying the nucleic acid sequences encoding the rAAV, thereby
producing the rAAV.

2. The method of claim 1, wherein cells of said cell line are transfected in suspension.

3. The method of claim 1, wherein the human embryonic cell line is suspension-adapted, serum-free cell line derived from a human embryonic kidney cell line.

4. The method of claim 1, wherein the AAV rep and AAV cap genes are from different serotypes.

5. The method of claim 1, wherein the AAV rep and AAV cap genes are from same serotypes.

6. The method of claim 1, wherein the AAV rep gene is an AAV2 rep gene and the AAV cap gene is an AAV8 cap gene.

7. The method of claim 1, wherein the AAV ITR and the AAV cap genes are from different serotypes.

8. The method of claim 1, wherein the AAV ITR and the AAV cap genes are from same serotype.

9. The method of claim 1, wherein the AAV inverted terminal repeat (ITR) sequences are adeno-associated virus 2 inverted terminal repeat (ITR) sequences.

10. The method of claim 1, wherein the AAV ITR sequence is from AAV 2 serotype or serotypes selected from the group consisting of AAV1, 3a, 3b, 4, 5, 6, 7, 8, 9, 10, 11, and 13.

11. The method of claim 1, wherein the AAV ITR sequence is synthetic.

12. The method of claim 1, wherein the total amount of nucleic acid transfected from a), b), and c) per 1 × 10⁶ cells is less than 2 µg, optionally, the total amount of nucleic acid transfected from a), b), and c) per 1 × 10⁶ cells is less than 1 µg.

13. The method of claim 1, wherein the ratio of a):b):c) is about 0.5-1.75: about 0.75-2.25: about 0.5-1.75 (weight:weight:weight), optionally, the ratio of a):b):c) is about 1:about 1-1.6: about 1 (weight:weight:weight).

14. The method of claim 1, wherein a), b) and c) are transfected using a transfection composition comprising a), b) and c), and a stable cationic polymer, wherein the ratio of stable cationic polymer to total amount of nucleic acid from a), b) and c), is from about 1:1 to about 3:1 (weight/weight), optionally, the ratio of stable cationic polymer to total amount of nucleic acid from a), b) and c), is about 1.5:1.

15. The method of claim 1, wherein each of a), b) and c) are provided on one or more close ended linear duplexed nucleic acid molecules.

16. The method of claim 1, wherein the transfected nucleic acids a), b), and c) are synthetic nucleic acids and devoid of eukaryotic and prokaryotic cellular modifications of DNA.

17. The method of claim 1, wherein the nucleic acid sequence encoding helper proteins sufficient for rAAV replication comprises a nucleotide sequence encoding an adenovirus helper (Ad helper) protein, optionally the nucleic acid sequence encoding helper proteins sufficient for rAAV replication comprises a nucleotide sequence encoding adenoviral helper proteins E2A and E4.

18. The method of claim 1, wherein the titer of rAAV is at least 9.3 x 10¹³ vector genomes/3.0 x 10⁹ viable cells transfected.

19. The method of claim 1, wherein the suspension of human embryonic cell line is progressively cultured in increasing volumes prior to transfection.

20. The method of claim 19, wherein the culturing volumes are progressively increased from about 50 ml volumes to about 2000 liters volume.

21. The method of claim 20, wherein the culturing volume comprises a concentration of an amino acid from about 1 mM to about 20 mM.

22. The method of claim 21, wherein the culture medium having a volume of about 5 liters comprises a concentration of an amino acid of about 10 mM.

23. The method of claims 21, wherein the amino acid is L-glutamine or L-alanyl-L-glutamine (Glutamax™).

24. The method of claim 20, wherein the culture medium having a volume of about 50 liters comprises: at least, from about 1 mM to about 20 mM L-glutamine, at least from about 0.01% to about 1% a nonionic, surfactant polyol or detergent and at least, from about 0.001% to about 1% of an anti-foaming agent.

25. The method of claim 24, wherein the nonionic, surfactant polyol comprises pluronic acid.

26. The method of claim 1, wherein the cultured human embryonic cell line comprises a cell density of about 3.0 ×10⁶ to about 1×10⁸ viable cells/ml.

27. The method of claim 26, wherein the cultured human embryonic cell line comprises a cell density of about 4.0 ×10⁶ to about 6× 10⁶ viable cells/ml or optionally to about 2.5 × 10⁷ viable cells/ml.

28. The method of claim 1, further comprising: (i) adding about 1 liter of medium to the transfected cells; and (ii) adding a cationic polymer at a ratio of between about 1:1 of polymer to DNA to about 3:1 of the polymer to DNA over a time course of about 10 minutes to about 60 minutes.

29. The method of claim 28, wherein the cationic polymer is added at a ratio of 2.2:1 of the polymer to DNA over a time course of about 1 minute to about 10 minutes.

30. The method of claim 28, wherein the cationic polymer comprises a fully hydrolyzed linear polyethylenimine (PEI).

31. The method of claim 1, wherein temperature of the culture medium comprising the human embryonic cell suspension is increased to 37° C. at about 12 to 36 hours prior to transfection.

32. The method of claim 27, wherein the culture medium is subjected to an air sparge at a flow rate between about 0.1 LPM to about 1.0 LPM.

33. The method of claim 27, wherein the culture medium is subjected to an air sparge at a flow rate of about 0.5 LPM.

34. A method of producing a population of high titer recombinant adeno-associated virus (rAAV) lacking prokaryotic sequences comprising:
i) transfecting a mammalian cell line with a) a nucleic acid sequence encoding helper proteins sufficient for rAAV replication; b) a nucleic acid sequence encoding rep and cap genes, and c) a close ended linear duplexed rAAV vector nucleic acid comprising at least one ITR and a heterologous transgene operably linked to one or more regulatory elements, wherein the total amount of nucleic acid transfected from a), b), and c) per 1 X 10⁶ cells is less than 1 µg;
ii) culturing the transfected cells for at least 24 hours;
iii) harvesting the transfected cells and purifying the rAAV vector particles produced;
wherein the titer of rAAV is at least 9.3 x 10¹³ vector genomes/3.0 x 10⁹ viable cells transfected.

35. The method of claim 34, wherein the mammalian cell line is a suspension cell line and the cells are transfected in suspension.

36. The method of claim 34, wherein the cell line is derived from a human embryonic kidney cell line.

37. The method of any of claims 34 through 36, wherein the mammalian cell line is a suspension adapted serum free cell line.

38. The method of claim 34, wherein the ratio of a):b):c) is about 0.5-1.75: about 0.75-2.25: about 0.5-1.75 (weight:weight:weight), optionally the ratio of a):b):c) is about 1:about 1-1.6: about 1 (weight:weight:weight).

39. The method of claim 34, wherein a), b) and c) are transfected using a transfection composition comprising a), b) and c), and a stable cationic polymer, wherein the ratio of stable cationic polymer to total amount of nucleic acid from a), b) and c), is from about 1:1 to about 3:1 (weight/weight), optionally, the ratio of stable cationic polymer to total amount of nucleic acid from a), b) and c), is about 1.5:1.

40. The method of claim 34, wherein each of a), b) and c) are provided on one or more close ended linear duplexed nucleic acid molecules.

41. The method of claim 34, wherein the transfected nucleic acids a), b), and c) are synthetic nucleic acids and devoid of eukaryotic and prokaryotic cellular modifications of DNA.

42. The method of claim 34, wherein a) the nucleic acid sequence encoding helper proteins sufficient for rAAV replication comprises a nucleotide sequence encoding an Ad helper protein, optionally the nucleic acid sequence encoding helper proteins sufficient for rAAV replication comprises a nucleotide sequence encoding adenoviral helper proteins E2A and E4.

43. The method of any of claims 34 through 42, wherein, the amount of total of DNA from a), b) and c) are optionally 0.6, 0.7, 0.75, 0.8, 0.9, 1, 1.2, 1.4, 1.6 or 1.8 µg.

44. The method of any of claims 34 through 43, wherein the suspension of the mammalian cell line is progressively cultured in increasing volumes of culture medium prior to transfection.

45. The method of claim 44, wherein the culturing volumes are progressively increased from about 50 ml volumes to about 2000 liter volumes.

46. The method of claim 45, wherein the culturing volume comprises a concentration of an amino acid from about 1 mM to about 20 mM.

47. The method of claim 46, wherein the culture medium having a volume of about 5 liters comprises a concentration of an amino acid of about 10 mM.

48. The method of claims 47, wherein the amino acid is L-glutamine.

49. The method of claim 48, wherein the cells are in a culture volume of 50 to 100 liters.

50. The method of any preceding claims wherein the infectious particle titer is at least 3 x 10⁹ TCID50/ml.

51. The method of claim 34, wherein the AAV Rep and the AAV Cap genes are from the same AAV serotype.

52. The method of claim 34, wherein the AAV Rep and the AAV Cap genes are from different AAV serotypes.

53. The method of claim 34, wherein the AAV ITR and the AAV Cap genes are from the same AAV serotype.

54. The method of claim 34, wherein the AAV ITR and the AAV Cap genes are from different AAV serotypes.

55. The method of claim 34, wherein the AAV ITR sequence is from AAV2 or from serotypes selected from the group consisting of AAV1, 3a, 3b, 4, 5, 6, 7, 8, 9, 10, 11, and 13.

56. The method of claim 34, wherein the AAV ITR sequence is synthetic.

57. A method of producing a population of purified recombinant adeno-associated virus (rAAV) that lacks prokaryotic sequences, comprising:
i. transfecting a mammalian cell line in suspended in culture medium with a transfection composition; wherein, the transfection composition comprises a) a nucleic acid sequence encoding helper proteins sufficient for rAAV replication ; b) a nucleic acid sequence encoding rep and cap genes, and c) a close ended linear duplexed rAAV vector nucleic acid comprising at least one ITR and a heterologous transgene operably linked to one or more regulatory elements, and d) a stable cationic polymer; and wherein,
the ratio of the stable cationic polymer to the total amount of nucleic acid contents from a), b) and c) is at least 1:1, optionally, the ratio of the stable cationic polymer to the total amount of nucleic acid contents from a), b) and c) is at least 1.5:1;
ii. culturing the transfected cell line for at least 24 hours;
iii. harvesting the transfected cell line of step ii);
iv. purifying the rAAV,
wherein the purified virus has a particle to infectivity ratio is less than 2 x 10⁴ vg/TCID50.

58. The method of claim 57, wherein the mammalian cell line is a suspension cell line and the cells are transfected in suspension.

59. The method of claim 57, wherein the mammalian cell line is derived from a human embryonic kidney cell line.

60. The method any of claims 57 through 59, wherein the human embryonic cell line is suspension-adapted, serum-free cell line derived from a human embryonic kidney cell line.

61. The method of claim 57, wherein the ratio of the stable cationic polymer to the total amount of nucleic acid contents from a), b) and c) is from about 1.75:1 to about 2.75:1, optionally, the ratio of the stable cationic polymer to the total amount of nucleic acid contents from a), b) and c) is about 2:1.

62. The method of any of claims 57 through 61, wherein the stable cationic polymer comprises a fully hydrolyzed linear polyethylenimine (PEI).

63. The method of claim 62, wherein the ratio of PEI to nucleic acid is selected from the group consisting of 1.2:1, 1.4:1, 1.6:1, 1.8:1, 2:1, 2.5:1, 2.8:1, and 2.2:1.

64. The method of claim 57, wherein temperature of the culture medium comprising the cell suspension is increased to 37° C. at about 12 to 36 hours prior to transfection.

65. The method of claim 57, wherein the culture medium is subjected to an air sparge at a flow rate between about 0.1 LPM to about 1.0 LPM.

66. The method of claim 65, wherein the culture medium is subjected to an air sparge at a flow rate of about 0.5 LPM.

67. The method of claim 57, wherein the transfection composition is added to the suspended cells over a time course of about 10 minutes to about 60 minutes.

68. The method of claim 57, wherein the culture medium is added after step i) and before step ii).

69. The method of claim 57, wherein the total amount of nucleic acid (DNA) from a), b) and c) is from about 1 µg to about 20 µg.

70. The method of claim 57, wherein the total amount of DNA from a), b) and c) is from about 1 µg to about 10 µg.

71. The method of claim 57, wherein the ratio of a):b):c) is about 0.5-1.75: about 0.75-2.25: about 0.5-1.75 (weight:weight:weight).

72. The method of claim 57, wherein each of a), b) and c) are provided on one or more close ended linear duplexed nucleic acid molecules.

73. The method of claim 57, wherein the transfected nucleic acids a), b), and c) are synthetic and devoid of eukaryotic and prokaryotic cellular modifications of DNA.

74. The method of claim 57, wherein the ratio of a):b):c) is about 1:about 1-1.6: about 1 (weight:weight:weight).

75. The method of claim 57, wherein a) the nucleic acid sequence encoding helper proteins sufficient for rAAV replication comprises a nucleotide sequence encoding an Ad helper protein, optionally, the nucleic acid sequence encoding helper proteins sufficient for rAAV replication comprises a nucleotide sequence encoding adenoviral helper proteins E2A and E4.

76. The method of claim 57, wherein the amount of total of DNA from a), b) and c) is 0.6, 0.7, 0.8, 0.9, 1, 1.2, 1.4, 1.6 or 1.8 µg.

77. The method of claim 76, wherein the amount of total of DNA from a), b) and c); is about 0.75 µg.

78. The method of claim 57, wherein the suspension of the mammalian cell line is progressively cultured in increasing volumes prior to transfection.

79. The method of claim 57, wherein the culturing volumes are progressively increased from about 50 ml volumes to about 2000 liter volumes.

80. The method of claim 57, wherein the culturing volumes are progressively increased from about 50 ml volumes to about 100 liter volumes.

81. The method of claim 79, wherein the culturing volume comprises a concentration of an amino acid from about 1 mM to about 20 mM.

82. The method of claim 79 or 80, wherein the culture medium having a volume of about 5 liters comprises a concentration of an amino acid of about 10 mM.

83. The method of claims 81 or 82, wherein the amino acid is L-glutamine.

84. The method of claim 83, wherein the cells are in a culture volume of 50 litres to 100 liters.

85. The method of claim 57, wherein the culture medium having a volume of about 50 liters comprises: at least, from about 1 mM to about 20 mM L-glutamine, at least from about 0.01% to about 1% a nonionic, surfactant polyol or detergent and at least, from about 0.001% to about 1% of an anti-foaming agent.

86. The method of claim 85, wherein the nonionic, surfactant polyol comprises pluronic acid.

87. The method of claim 57, wherein the transfection composition comprises at least about 5% volume/volume (v/v) to about 20% v/v of the culture medium.

88. The method of claim 57, wherein the transfection composition comprises about 1 liter to about 5 liters of medium.

89. The method of claim 57, wherein the transfection composition comprises 5-50% (volume/volume) of culture medium.

90. The method of claim 57, wherein the nucleic acid sequences added to the transfection comprise: about 0.1 µg to about 1 µg of Ad helper DNA, Rep/Cap DNA, or transgene per 0.5 ×10⁶ to about 5 ×10⁶ cells.

91. The method of any of claims 1, 34 or 57, wherein the packaged nucleic acid of the rAAV further lacks eukaryotic DNA sequences.

92. The method of claim 57, wherein the AAV Rep and the AAV Cap genes are from same AAV serotype.

93. The method of claim 57, wherein, the AAV Rep and the AAV Cap genes are from different AAV serotypes.

94. The method of claim 57, wherein the AAV ITR and the AAV Cap genes are from same AAV serotype.

95. The method of claim 57, wherein the AAV ITR and the AAV Cap genes are from different AAV serotypes.

96. The method of claim 57, wherein the AAV ITR sequence is from AAV2 or from serotypes selected from the group consisting of AAV1, 3a, 3b, 4, 5, 6, 7, 8, 9, 10, 11, and 13.

97. The method of claim 57, wherein the Cap gene is from AAV8 serotype.

98. The method of claim 57, wherein the closed ended linear duplexed nucleic acid comprises ½ of a protelomerase binding site.

99. The method of claim 98, wherein the ½ of a protelomerase binding site is formed by protelomerase digestion of a target binding site comprising a double stranded palindromic sequence of at least 10 base pairs in length.

100. A population of rAAV virions that lack prokaryotic DNA produced by the method of any one of claims 1, 34 or 57.

101. A recombinant adeno-associated virus (rAAV) comprising a protelomerase target sequence.

102. The rAAV of claim 101, wherein the protelomerase target sequence comprises a double stranded palindromic sequence of at least 10 base pairs in length.

103. The rAAV of claim 101, further comprising a transgene.
